(19) United States
(12) Patent Application Publication
(10) Pub. No.: US 2025/0277216 A1
(43) Pub. Date: Sep. 4, 2025

(54) Allele-Specific Silencing of Transforming Growth Factor Beta Induced Gene with R124H Mutation Using Short Interfering RNA

(71) Applicant: Avellino Lab USA, Inc., Menlo Park, CA (US)

(72) Inventors: Tara Moore, Ballyclare (GB); Amanda Kowalczyk, Menlo Park, CA (US)

(73) Assignee: Avellino Lab USA, Inc., Menlo Park (CA)

(21) Appl. No.: 17/784,878

(22) PCT Filed: Dec. 11, 2020
(86) PCT No.: PCT/IB2020/061852

§ 371 Date: Jun. 13, 2022

Related U.S. Application Data
(60) Provisional application No. 62/946,880, filed on Dec. 11, 2019.
(60) Provisional application No. 62/975,713, filed on Feb. 12, 2020.

(51) Int. Cl. C12N 15/113

(52) U.S. Cl. CPC C12N 15/1136; C12N 2310/11; C12N 2310/14; C12N 2310/321; C12N 2310/322; C12N 2320/32

(57) ABSTRACT

The present disclosure relates to a ribonucleic acid (RNA) complex and methods of use thereof for preventing, amelio-rating or treating symptoms associated with granular corneal dystrophy type 2 in a subject.

### FIELD

The present disclosure relates generally to small interfering ribonucleic acid (siRNA) mediated inhibition of gene expression, and in particular to, methods and compositions for inhibiting expression of mutant transforming growth factor beta induced (TGFBI) protein in a subject.

### BACKGROUND

Ribonucleic acid (RNA) plays various roles in living cells. In particular, messenger RNA (mRNA) molecules carry genetic information from deoxyribonucleic acid (DNA) to enable synthesis of proteins. RNA interference (RNAi) has been shown as a way to inhibit or reduce expression of particular genes, such as disease-causing genes. Disorders treated by RNAi therapeutics in clinical trials include pachyonychia congenita, age-related macular degeneration, hepatitis C, and chronic myeloid leukemia (Davidson B L, McCray P B. Current prospects for RNA interference-based therapies. Nat Rev Genet. 2011; 12: 329-340).

The cornea is an avascular, transparent tissue found in the anterior segment of the eye. The main function of the cornea is to act as a structural barrier to the outside and provide the majority of the eye's refractive power. The cornea is divided into five layers: epithelium, Bowman's layer, stroma, Descemet's membrane, and endothelium.

The TGFBI gene is located in cytogenetic band 5q31.1. Mutations in the TGFBI gene cause a range of corneal dystrophies (Munier F L, Frueh B E, Othenin-Girard P, et al. BIGH3 mutation spectrum in corneal dystrophies. Invest Ophthalmol Vis Sci. 2002; 43: 949-954), including Granular Corneal Dystrophy Type I, Granular Corneal Dystrophy Type II, Lattice Corneal Dystrophy Type I, Thiel-Behnke Corneal Dystrophy, and Reis-bucklers Corneal Dystrophy. These corneal dystrophies can lead to excess accumulation of TGFBI protein in the cornea, which results in impaired vision.

Conventional treatments for such corneal dystrophies are laser resurfacing keratectomy and surgical keratoplasty, an invasive procedure where pathologically affected corneal tissue is excised (either full thickness or partial thickness) and replaced by transplanted donor tissue. These treatments are only partially effective, require long-term monitoring follow-up, and can be associated with various morbidities. In particular, these treatments can induce increased expression of mutant TGFBI proteins due to the corneal injury caused during the treatment, which usually leads to recurrence of the impaired vision.

Similarly, laser-assisted in situ kertomileusis (LASIK) surgery, which also causes corneal injury, can accelerate the vision impairment by triggering the excess accumulation of mutant TGFBI proteins. In particular, it has been observed that heterozygous individuals, who typically would have a late onset of vision impairment in the absence of LASIK surgery, start to have accelerated loss of vision following LASIK surgery (Jun, R. M. et al., Ophthalmology, 111:463, 2004).

Corneal dystrophy can be an autosomal dominant hereditary disease. Thus, a heterozygous individual, who having one wild type TGFBI allele and one mutant TGFBI allele, can suffer from the corneal dystrophy. However, silencing both the wild type TGFBI allele and the mutant TGFBI allele inhibits expression of wild type TGFBI protein, which plays vital roles in cells, such as modulating cell adhesion and corneal wound healing.

### SUMMARY

Thus, allele-specific silencing of the mutant TGFBI allele is required for maintaining the vitality of cells and treating, reducing, and preventing corneal dystrophy associated with the mutant TGFBI. Such allele-specific silencing of the mutant TGFBI allele is achieved by one or more RNA complexes disclosed herein.

In accordance with some embodiments, a ribonucleic acid (RNA) complex includes a strand that includes a sequence having at least 80% identity to one of SEQ ID NOs: 1-19.

In some embodiments of each or any of the above or below mentioned embodiments, the strand comprises a sequence having at least 80% identity to SEQ ID NO: 4.

In some embodiments of each or any of the above or below mentioned embodiments, the strand includes at least one of: an overhang at the 5′ end of the sequence having at least 80% identity to SEQ ID NO: 4 or an overhang at the 3′ end of the sequence having at least 80% identity to SEQ ID NO: 4.

In some embodiments of each or any of the above or below mentioned embodiments, the strand includes at least one of: cytidine monophosphate (rC) at the 5′ end of the sequence having at least 80% identity to SEQ ID NO: 4, uridine monophosphate-cytidine monophosphate (rU-rC) at the 5′ end of the sequence having at least 80% identity to SEQ ID NO: 4, double uridine monophosphate (rU-rU) at the 3′ end of the sequence having at least 80% identity to SEQ ID NO: 4, double deoxythymidine monophosphates (dT-dT) at the 3′ end of the sequence having at least 80% identity to SEQ ID NO: 4, double 2′-O-methylated guanosine monophosphates (oG-oG) at the 3′ end of the sequence having at least 80% identity to SEQ ID NO: 4, and double 2′-O-methyl-uridine monophosphates (oU-oU) at the 3′ end of the sequence having at least 80% identity to SEQ ID NO: 4.

In some embodiments of each or any of the above or below mentioned embodiments, the RNA complex further comprises a strand that comprises a sequence having at least 80% identity to SEQ ID NO: 23.

In some embodiments of each or any of the above or below mentioned embodiments, the RNA complex further comprises a strand that comprises a sequence of SEQ ID NO: 23.

In some embodiments of each or any of the above or below mentioned embodiments, the RNA complex further comprises a strand that consists of a sequence of SEQ ID NO: 23.

In some embodiments of each or any of the above or below mentioned embodiments, the strand comprises a sequence of SEQ ID NO: 4.

In some embodiments of each or any of the above or below mentioned embodiments, the strand includes at least one of: an overhang at the 5′ end of the sequence of SEQ ID NO: 4 or an overhang at the 3′ end of the sequence of SEQ ID NO: 4.

In some embodiments of each or any of the above or below mentioned embodiments, the strand includes at least one of: cytidine monophosphate (rC) at the 5′ end of the sequence of SEQ ID NO: 4, uridine monophosphate-cytidine monophosphate (rU-rC) at the 5′ end of the sequence of SEQ ID NO: 4, double uridine monophosphate (rU-rU) at the 3′ end of the sequence of SEQ ID NO: 4, double deoxythymidine monophosphates (dT-dT) at the 3′ end of the sequence of SEQ ID NO: 4, double 2′-O-methylated guanosine monophosphates (oG-oG) at the 3′ end of the sequence of SEQ ID NO: 4, and double 2′-O-methyl-uridine monophosphates (oU-oU) at the 3′ end of the sequence of SEQ ID NO: 4.

In some embodiments of each or any of the above or below mentioned embodiments, the RNA complex comprises a first strand and a second strand, each comprising at least one TGFBI R124H mutation site compared to a wild-type 124C TGFBI gene.

In some embodiments of each or any of the above or below mentioned embodiments, the first strand comprises a sequence that is identical to the sequence of SEQ ID NO: 4, except for one base that is mismatched to a juxtaposed base in the second strand.

In some embodiments of each or any of the above or below mentioned embodiments, the first strand and the second strand each comprise one TGFBI R124H mutation site.

In some embodiments of each or any of the above or below mentioned embodiments, the mismatched base is between three and seven bases away from the TGFBI R124H mutation site.

In some embodiments of each or any of the above or below mentioned embodiments, the mismatched base is three bases away from the TGFBI R124H mutation site.

In some embodiments of each or any of the above or below mentioned embodiments, the mismatched base is four bases away from the TGFBI R124H mutation site.

In some embodiments of each or any of the above or below mentioned embodiments, the mismatched base is five bases away from the TGFBI R124H mutation site.

In some embodiments of each or any of the above or below mentioned embodiments, the mismatched base is six bases away from the TGFBI R124H mutation site.

In some embodiments of each or any of the above or below mentioned embodiments, the mismatched base is seven bases away from the TGFBI R124H mutation site.

In some embodiments of each or any of the above or below mentioned embodiments, the first strand and the second strand are each between 16 and 23 bases in length.

In some embodiments of each or any of the above or below mentioned embodiments, the first strand and the second strand are each 22 bases in length.

In some embodiments of each or any of the above or below mentioned embodiments, the RNA complex has deoxythymidine overhangs.

In some embodiments of each or any of the above or below mentioned embodiments, the first strand comprises a sequence that is identical to the sequence of SEQ ID NO: 9, except for one base that is mismatched to a juxtaposed base in the second strand.

In some embodiments of each or any of the above or below mentioned embodiments, the first strand and the second strand each comprise one TGFBI R124H mutation site.

In some embodiments of each or any of the above or below mentioned embodiments, the mismatched base is between three and seven bases away from the TGFBI R124H mutation site.

In some embodiments of each or any of the above or below mentioned embodiments, the mismatched base is three bases away from the TGFBI R124H mutation site.

In some embodiments of each or any of the above or below mentioned embodiments, the mismatched base is four bases away from the R124H mutation site.

In some embodiments of each or any of the above or below mentioned embodiments, the mismatched base is five bases away from the R124H mutation site.

In some embodiments of each or any of the above or below mentioned embodiments, the mismatched base is six bases away from the R124H mutation site.

In some embodiments of each or any of the above or below mentioned embodiments, the mismatched base is seven bases away from the R124H mutation site.

In some embodiments of each or any of the above or below mentioned embodiments, the first strand and the second strand are each between 16 and 23 bases in length.

In some embodiments of each or any of the above or below mentioned embodiments, the first strand and the second strand are each 22 bases in length.

In some embodiments of each or any of the above or below mentioned embodiments, the RNA complex has deoxythymidine overhangs.

In some embodiments of each or any of the above or below mentioned embodiments, the first strand comprises a sequence that is identical to the sequence of SEQ ID NO: 11, except for one base that is mismatched to a juxtaposed base in the second strand.

In some embodiments of each or any of the above or below mentioned embodiments, the first strand and the second strand each comprise one R124H mutation site.

In some embodiments of each or any of the above or below mentioned embodiments, the mismatched base is between three and seven bases away from the R124H mutation site.

In some embodiments of each or any of the above or below mentioned embodiments, the mismatched base is three bases away from the R124H mutation site.

In some embodiments of each or any of the above or below mentioned embodiments, the mismatched base is four bases away from the R124H mutation site.

In some embodiments of each or any of the above or below mentioned embodiments, the mismatched base is five bases away from the R124H mutation site.

In some embodiments of each or any of the above or below mentioned embodiments, the mismatched base is six bases away from the R124H mutation site.

In some embodiments of each or any of the above or below mentioned embodiments, the mismatched base is seven bases away from the R124H mutation site.

In some embodiments of each or any of the above or below mentioned embodiments, the first strand and the second strand are each between 16 and 23 bases in length.

In some embodiments of each or any of the above or below mentioned embodiments, the first strand and the second strand are each 22 bases in length.

In some embodiments of each or any of the above or below mentioned embodiments, the RNA complex has deoxythymidine overhangs.

In some embodiments of each or any of the above or below mentioned embodiments, the RNA complex further comprises a strand that comprises a sequence having at least 80% identity to SEQ ID NO: 23.

In some embodiments of each or any of the above or below mentioned embodiments, the RNA complex further comprises a strand that comprises a sequence of SEQ ID NO: 23.

In some embodiments of each or any of the above or below mentioned embodiments, the RNA complex further comprises a strand that consists of a sequence of SEQ ID NO: 23.

In some embodiments of each or any of the above or below mentioned embodiments, the strand consists of a sequence of SEQ ID NO: 4.

In some embodiments of each or any of the above or below mentioned embodiments, the RNA complex further comprises a strand that comprises a sequence having at least 80% identity to SEQ ID NO: 23.

In some embodiments of each or any of the above or below mentioned embodiments, the RNA complex further comprises a strand that comprises a sequence of SEQ ID NO: 23.

In some embodiments of each or any of the above or below mentioned embodiments, the RNA complex further comprises a strand that consists of a sequence of SEQ ID NO: 23.

In some embodiments of each or any of the above or below mentioned embodiments, wherein the strand comprises a sequence having at least 80% identity to SEQ ID NO: 11.

In some embodiments of each or any of the above or below mentioned embodiments, the strand includes at least one of: an overhang at the 5′ end of the sequence having at least 80% identity to SEQ ID NO: 11 or an overhang at the 3′ end of the sequence having at least 80% identity to SEQ ID NO: 11.

In some embodiments of each or any of the above or below mentioned embodiments, the strand includes at least one of: adenosine monophosphate (rA) at the 5′ end of the sequence having at least 80% identity to SEQ ID NO: 11, uridine monophosphate-adenosine monophosphate (rU-rA) at the 5′ end of the sequence having at least 80% identity to SEQ ID NO: 11, double uridine monophosphate (rU-rU) at the 3′ end of the sequence having at least 80% identity to SEQ ID NO: 11, double deoxythymidine monophosphates (dT-dT) at the 3′ end of the sequence having at least 80% identity to SEQ ID NO: 11, 2′-O-methylated cytidine monophosphate-uridine monophosphate (oC-oU) at the 3′ end of the sequence having at least 80% identity to SEQ ID NO: 11, and double 2′-O-methyl-uridine monophosphates (oU-oU) at the 3′ end of the sequence having at least 80% identity to SEQ ID NO: 11.

In some embodiments of each or any of the above or below mentioned embodiments, the RNA complex further comprises a strand that comprises a sequence having at least 80% identity to SEQ ID NO: 30.

In some embodiments of each or any of the above or below mentioned embodiments, the RNA complex further comprises a strand that comprises a sequence of SEQ ID NO: 30.

In some embodiments of each or any of the above or below mentioned embodiments, the RNA complex further comprises a strand that consists of a sequence of SEQ ID NO: 30.

In some embodiments of each or any of the above or below mentioned embodiments, the strand comprises a sequence of SEQ ID NO: 11.

In some embodiments of each or any of the above or below mentioned embodiments, the strand includes at least one of: an overhang at the 5′ end of the sequence of SEQ ID NO: 11 or an overhang at the 3′ end of the sequence of SEQ ID NO: 11.

In some embodiments of each or any of the above or below mentioned embodiments, the strand includes at least one of: adenosine monophosphate (rA) at the 5′ end of the sequence of SEQ ID NO: 11, uridine monophosphate-adenosine monophosphate (rU-rA) at the 5′ end of the sequence of SEQ ID NO: 11, double uridine monophosphate (rU-rU) at the 3′ end of the sequence of SEQ ID NO: 11, double deoxythymidine monophosphates (dT-dT) at the 3′ end of the sequence of SEQ ID NO: 11, 2′-O-methylated cytidine monophosphate-uridine monophosphate (oC-oU) at the 3′ end of the sequence of SEQ ID NO: 11, and double 2′-O-methyl-uridine monophosphates (oU-oU) at the 3′ end of the sequence of SEQ ID NO: 11.

In some embodiments of each or any of the above or below mentioned embodiments, the RNA complex further comprises a strand that comprises a sequence having at least 80% identity to SEQ ID NO: 30.

In some embodiments of each or any of the above or below mentioned embodiments, the RNA complex further comprises a strand that comprises a sequence of SEQ ID NO: 30.

In some embodiments of each or any of the above or below mentioned embodiments, the RNA complex further comprises a strand that consists of a sequence of SEQ ID NO: 30.

In some embodiments of each or any of the above or below mentioned embodiments, the strand consists of a sequence of SEQ ID NO: 11.

In some embodiments of each or any of the above or below mentioned embodiments, the RNA complex further comprises a strand that comprises a sequence having at least 80% identity to SEQ ID NO: 30.

In some embodiments of each or any of the above or below mentioned embodiments, the RNA complex further comprises a strand that comprises a sequence of SEQ ID NO: 30.

In some embodiments of each or any of the above or below mentioned embodiments, the RNA complex further comprises a strand that consists of a sequence of SEQ ID NO: 30.

In some embodiments of each or any of the above or below mentioned embodiments, the strand comprises a sequence having at least 80% identity to SEQ ID NO: 16.

In some embodiments of each or any of the above or below mentioned embodiments, the strand includes at least one of: an overhang at the 5′ end of the sequence having at least 80% identity to SEQ ID NO: 16 or an overhang at the 3′ end of the sequence having at least 80% identity to SEQ ID NO: 16.

In some embodiments of each or any of the above or below mentioned embodiments, the strand includes at least one of: guanosine monophosphate (rG) at the 5′ end of the sequence having at least 80% identity to SEQ ID NO: 16, double guanosine monophosphate (rG-rG) at the 5′ end of the sequence having at least 80% identity to SEQ ID NO: 16, double uridine monophosphate (rU-rU) at the 3′ end of the sequence having at least 80% identity to SEQ ID NO: 16, double deoxythymidine monophosphates (dT-dT) at the 3′ end of the sequence having at least 80% identity to SEQ ID NO: 16, 2′-O-methylated guanosine monophosphate-cytidine monophosphate (oG-oC) at the 3′ end of the sequence having at least 80% identity to SEQ ID NO: 16, and double 2′-O-methyl-uridine monophosphates (oU-oU) at the 3′ end of the sequence having at least 80% identity to SEQ ID NO: 16.

In some embodiments of each or any of the above or below mentioned embodiments, the RNA complex further comprises a strand that comprises a sequence having at least 80% identity to SEQ ID NO: 35.

In some embodiments of each or any of the above or below mentioned embodiments, the RNA complex further comprises a strand that comprises a sequence of SEQ ID NO: 35.

In some embodiments of each or any of the above or below mentioned embodiments, the RNA complex further comprises a strand that consists of a sequence of SEQ ID NO: 35.

In some embodiments of each or any of the above or below mentioned embodiments, the strand comprises a sequence of SEQ ID NO: 16.

In some embodiments of each or any of the above or below mentioned embodiments, the strand includes at least one of: an overhang at the 5′ end of the sequence of SEQ ID NO: 16 or an overhang at the 3′ end of the sequence of SEQ ID NO: 16.

In some embodiments of each or any of the above or below mentioned embodiments, the strand includes at least one of: guanosine monophosphate (rG) at the 5′ end of the sequence of SEQ ID NO: 16, double guanosine monophosphate (rG-rG) at the 5′ end of the sequence of SEQ ID NO: 16, double uridine monophosphate (rU-rU) at the 3′ end of the sequence of SEQ ID NO: 16, double deoxythymidine monophosphates (dT-dT) at the 3′ end of the sequence of SEQ ID NO: 16, 2′-O-methylated guanosine monophosphate-cytidine monophosphate (oG-oC) at the 3′ end of the sequence of SEQ ID NO: 16, and double 2′-O-methyl-uridine monophosphates (oU-oU) at the 3′ end of the sequence of SEQ ID NO: 16.

In some embodiments of each or any of the above or below mentioned embodiments, the RNA complex further comprises a strand that comprises a sequence having at least 80% identity to SEQ ID NO: 35.

In some embodiments of each or any of the above or below mentioned embodiments, the RNA complex further comprises a strand that comprises a sequence of SEQ ID NO: 35.

In some embodiments of each or any of the above or below mentioned embodiments, the RNA complex further comprises a strand that consists of a sequence of SEQ ID NO: 35.

In some embodiments of each or any of the above or below mentioned embodiments, the strand consists of a sequence of SEQ ID NO: 16.

In some embodiments of each or any of the above or below mentioned embodiments, the RNA complex further comprises a strand that comprises a sequence having at least 80% identity to SEQ ID NO: 35.

In some embodiments of each or any of the above or below mentioned embodiments, the RNA complex further comprises a strand that comprises a sequence of SEQ ID NO: 35.

In some embodiments of each or any of the above or below mentioned embodiments, the RNA complex further comprises a strand that consists of a sequence of SEQ ID NO: 35.

In accordance with some embodiments, a ribonucleic acid (RNA) complex includes a strand that includes a sequence having at least 80% identity to one of SEQ ID NOs: 20-38.

In some embodiments of each or any of the above or below mentioned embodiments, wherein the strand comprises a sequence having at least 80% identity to SEQ ID NO: 23.

In some embodiments of each or any of the above or below mentioned embodiments, the strand includes at least one of: an overhang at the 5′ end of the sequence having at least 80% identity to SEQ ID NO: 23 or an overhang at the 3′ end of the sequence having at least 80% identity to SEQ ID NO: 23.

In some embodiments of each or any of the above or below mentioned embodiments, the strand comprises a sequence having at least 80% identity to SEQ ID NO: 30.

In some embodiments of each or any of the above or below mentioned embodiments, the strand includes at least one of: an overhang at the 5′ end of the sequence having at least 80% identity to SEQ ID NO: 30 or an overhang at the 3′ end of the sequence having at least 80% identity to SEQ ID NO: 30.

In some embodiments of each or any of the above or below mentioned embodiments, the strand comprises a sequence having at least 80% identity to SEQ ID NO: 35.

In some embodiments of each or any of the above or below mentioned embodiments, the strand includes at least one of: an overhang at the 5′ end of the sequence having at least 80% identity to SEQ ID NO: 35 or an overhang at the 3′ end of the sequence having at least 80% identity to SEQ ID NO: 35.

In accordance with some embodiments, a ribonucleic acid (RNA) complex includes a strand having a sequence overlapping with a sequence of a transforming growth factor beta induced (TGFBI) protein messenger RNA (mRNA), the strand containing adenine at a position corresponding to the c. 371G>A single nucleotide polymorphism (SNP) in exon 4 of the TGFBI gene.

In accordance with some embodiments, a ribonucleic acid (RNA) complex includes a sense strand and an antisense strand, wherein at least one of the sense strand and the antisense strand includes a dTdT overhang.

In some embodiments of each or any of the above or below mentioned embodiments, the sense strand and the antisense strand each comprises a dTdT overhang.

In some embodiments of each or any of the above or below mentioned embodiments, the sense strand comprises GG nucleotides as an overhang and the antisense strand comprises UC nucleotides as an overhang.

In some embodiments of each or any of the above or below mentioned embodiments, (a) the sense strand comprises a series of repeating 2′-OMe; and (b) the antisense strand comprises 2′-OMe.

In some embodiments of each or any of the above or below mentioned embodiments, (a) the sense strand comprises 15 bases and alternating patterns of 2′-OMe and 2′-F; and (b) the antisense strand comprises alternating patterns of 2′-OMe and 2′-F; wherein the RNA complex comprises additional phosphorothioate links on the 3′ and 5′ ends of both the sense strand and the antisense strand.

In some embodiments of each or any of the above or below mentioned embodiments, (a) the sense strand comprises two units of 2′-OMe at the 5′ end and at least two 2′-OMe modifications at either U or G residues other than at position 9; and (b) the antisense strand comprises a single 2′-OMe at position 2 from the 5′ end, PS bonds in a dTdT overhand, and all pyrimidines replaced with 2′F-RNA units.

In some embodiments of each or any of the above or below mentioned embodiments, the RNA complex includes a short interfering RNA duplex.

In some embodiments of each or any of the above or below mentioned embodiments, the RNA complex includes a double stranded RNA complex configured for forming a short interfering RNA duplex.

In some embodiments of each or any of the above or below mentioned embodiments, the RNA complex includes an RNA hairpin.

In accordance with some embodiments, a method of preventing, ameliorating, or treating granular corneal dystrophy type 2 in a subject includes administering to the subject any RNA complex described herein.

In some embodiments of each or any of the above or below mentioned embodiments, the administering comprises injecting the RNA complex into the subject.

In some embodiments of each or any of the above or below mentioned embodiments, the administering comprises applying a solution containing the RNA complex onto the subject.

In some embodiments of each or any of the above or below mentioned embodiments, the administering comprises introducing the RNA complex into a cell containing and expressing a deoxyribonucleic acid (DNA) molecule having the c. 371G>A single nucleotide polymorphism (SNP) in exon 4 of a transforming growth factor beta induced (TGFBI) gene.

In some embodiments of each or any of the above or below mentioned embodiments, the subject is a vertebrate.

In some embodiments of each or any of the above or below mentioned embodiments, the subject is human.

In some embodiments of each or any of the above or below mentioned embodiments, the method further comprises: prior to administering to the subject the RNA complex: obtaining sequence information of the subject; and determining that the subject has an allele having the c. 371G>A SNP in exon 4 of the TGFBI gene and an allele that does not have the c. 371G>A SNP in exon 4 of the TGFBI gene.

In some embodiments of each or any of the above or below mentioned embodiments, the sequence information of the subject consists of sequence information of exon 4 of the TGFBI gene.

In some embodiments of each or any of the above or below mentioned embodiments, the sequence information of the subject includes sequence information of a subset, less than all, of exon 4 of the TGFBI gene.

In some embodiments of each or any of the above or below mentioned embodiments, the sequence information of the subject includes only the sequence information of the c. 371G>A SNP in exon 4 of the TGFBI gene.

In some embodiments of each or any of the above or below mentioned embodiments, the sequence information of the subject includes whole-genome sequence information of the subject.

### BRIEF DESCRIPTION OF THE DRAWINGS

The foregoing summary, as well as the following detailed description of the disclosure, will be better understood when read in conjunction with the appended figures. For the purpose of illustrating the disclosure, shown in the figures are embodiments which are presently preferred. It should be understood, however, that the disclosure is not limited to the precise arrangements, examples and instrumentalities shown.

FIGS. 1A-1U show the effect of various wildtype (blue) and mutant (circled line) siRNA on luciferase expression 24 hours after transfection, using siLUC (FIG. 1A), NSC4 (FIG. 1B), siRNA1/SEQ ID NO: 1 (FIG. 1C), siRNA2/SEQ ID NO: 2 (FIG. 1D), siRNA3/SEQ ID NO: 3 (FIG. 1E), siRNA4/SEQ ID NO: 4 (FIG. 1F), siRNA5/SEQ ID NO: 5 (FIG. 1G), siRNA6/SEQ ID NO: 6 (FIG. 1H), siRNA7/SEQ ID NO: 7 (FIG. 1I), siRNA8/SEQ ID NO: 8 (FIG. 1J), siRNA9/SEQ ID NO: 9 (FIG. 1K), siRNA10/SEQ ID NO: 10 (FIG. 1L), siRNA11/SEQ ID NO: 11 (FIG. 1M), siRNA12/SEQ ID NO: 12 (FIG. 1N), siRNA13/SEQ ID NO: 13 (FIG. 1O), siRNA14/SEQ ID NO: 14 (FIG. 1P), siRNA15/SEQ ID NO: 15 (FIG. 1Q), siRNA16/SEQ ID NO: 16 (FIG. 1R), siRNA17/SEQ ID NO: 17 (FIG. 1S), siRNA18/SEQ ID NO: 18 (FIG. 1T), and siRNA19/SEQ ID NO: 19 (FIG. 1U). The Dual-Luciferase Reporter Assay (Promega, Southampton, UK) was used to measure luciferase expression according to the manufacturer's instructions, wherein first the medium was removed and cells were washed with PBS before replacement with passive lysis buffer (Promega), and second, cells were shaken on a plate shaker for 15 minutes to ensure they were fully lysed, before the activities of both firefly and Renilla luciferase were measured sequentially using the LUMIstar OPTIMA (BMG Labtech, Aylesbury, UK).

FIG. 2 shows siRNA sequences, with the R124H mutation in red and the mismatch introduced to the siRNA sequence highlighted in yellow.

FIG. 3 shows siRNA sequences of various lengths, with siRNA4 serving as the baseline for the length modifications, wherein ‘5’ or ‘3’ denotes the end where nucleotides were added (+) or removed (−) from the baseline sequence, ‘−n−n’ denotes the number of base pairs removed from both ends, and ‘+n+n’ denotes additions at both ends.

FIG. 4 shows top 5 siRNA with modified lengths at 0.25 nM dose. Results in quadruplicate were averaged and normalised to untreated wells. Blue bars represent knockdown of wild-type (wt) TGFBI variant and red bars are knockdown of disease-causing mutant-type (mut), with standard error bars and data table included.

FIG. 5 shows top 5 siRNA with modified lengths at 6.25 nM dose. Results in quadruplicate were averaged and normalised to untreated wells. Blue bars represent knockdown of wild-type (wt) allele and red bars are knockdown of disease-causing mutant-type (mut) allele, with standard error bars and data table included.

FIG. 6 shows average knockdown at lnM dose, normalised to untreated wells. Results in quadruplicate were averaged and normalised to untreated wells. Gray bars represent knockdown of luc2 plasmid, with standard error bars and data table included.

FIG. 7 shows average knockdown of luc2 plasmid in octuplicate replicates, which were normalised to untreated wells (0 nM). Doses used were: 0.1 nM to 10 nM. In green is indicated the response curve for chemically unmodified siLuc-dTdT and in purple the siLuc-mod3 response was plotted. Error bars showing standard deviation are included.

FIG. 8 shows combined gel electrophoresis demonstrating gradual degradation of siRNA variants from 0 to 72 hours caused by nucleases found in foetal bovine serum, with the stability assay prepared in duplicate.

FIG. 9 shows additional gel electrophoresis data for siRNA variants at the final 78h timepoint.

FIG. 10 shows average luciferase activity for siRNA 11-mismatch 2, the second candidate siRNA. The plot is an average of 8 wells; quadruplicate replicates run on two separate occasions with standard error bars included and activity normalised to untreated wells. Blue line represents activity of healthy wild-type allele whereas circled line represents activity of the disease-causing mutant-type allele.

FIGS. 11A-11C show average luciferase activity for siRNA4 (FIG. 11A), siRNA9 (FIG. 11B), and siRNA11 (FIG. 11C). Each graph represents an average of 8 wells; quadruplicate replicates run on two separate occasions with standard error bars included and activity normalised to untreated wells. Blue bars represent knockdown of healthy wild-type allele whereas orange bars with circles represent knockdown of the disease-causing mutant-type allele. Negative values mean that there was no knockdown effect on the wild-type plasmid. Mismatch siRNA were screened in 2 doses: 0.25 nM (left panels) and 6.25 nM (right panels).

FIGS. 12A and 12B show average knockdown by siRNA4 with modified lengths as compared to untreated, at two doses: 0.25 nM (FIG. 12A) and 6.25 nM (FIG. 12B). Each graph represents an average of four replicates with standard error bars included and activity normalised to untreated wells. Blue bars represent knockdown of healthy wild-type allele (wt) whereas orange bars with circles represent knockdown of the disease-causing mutant-type allele (mut).

### DETAILED DESCRIPTION

As used throughout, ranges are used as shorthand for describing each and every value that is within the range. Any value within the range can be selected as the terminus of the range. In addition, all references cited herein are hereby incorporated by reference in their entireties for all purposes. In the event of a conflict in a definition in the present disclosure and that of a cited reference, the present disclosure controls.

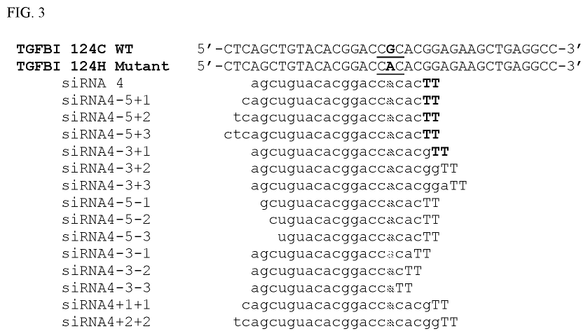

Ribonucleic acids (RNA) play a vital role in expression of genes. RNA interference (RNAi) is a biological mechanism where double-stranded RNA (dsRNA) molecules silence or knockdown the post-transcriptional expression of the target genes. Short interfering RNA (siRNA) are synthetic mediators of RNAi mechanism. They are dsRNA molecules typically containing 21-23 base pairs and are specifically designed to silence expression of target genes. siRNA may be introduced exogenously into the cell in a short form (already as a siRNA duplex) or in a form of long dsRNA molecules, which are processed within the cells (e.g., by a dicer enzyme) and converted into siRNAs. Dicer enzymes typically leave 2 nucleotide overhangs in 3′ direction and phosphate group in the 5′ direction. siRNAs are then recognized by RISC-Ago2 enzyme complex. One of the siRNA strands is degraded and the antisense strands acts as guide for the RISC complex to find the correct mRNA sequence that requires silencing (FIG. 3).

In accordance with some embodiments, a ribonucleic acid (RNA) complex includes a strand (e.g., a sense strand) that includes a sequence having at least 80% identity (e.g., 80%, 82%, 84%, 85%, 86%, 88%, 90%, 92%, 94%, 95%, 96%, 98%, 99%, or 100%) to one of SEQ ID NOs: 1-19. In some embodiments, the strand has a single nucleotide mismatch adjacent to the mutation site as shown below (e.g., 3-base-pairs, 5-base-pairs, or 7-base-pairs away from the mutation site):

| TGFBI 124C WT | | 5′-CTCAGCTGTACACGGACCG |
|---|---|---|
| | | CACGGAGAAGCTGAGGCC-3′ |
| TGFBI 124H | | 5′-CTCAGCTGTACACGGACCA |
| Mutant | | CGGAGAAGCTGAGGCC-3′ |
| 3 bp away | 9 | uacacggaccacaGggagaTT |
| | 11 | cacggaccacaGggagaagTT |
| 5 bp away | 9 | uacacggaccacacgCagaTT |
| | 11 | cacggaccacacgCagaagTT |
| 7 bp away | 9 | uacacggaccacacggaCaTT |
| | 11 | cacggaccacacggaCaagTT |

In some embodiments, the RNA complex described herein may comprise a first strand and a second strand, each comprising at least one or one mutation site corresponding to a site in TGFBI gene compared to a wild-type TGFBI. Exemplary TGFBI mutations are described in Yamazoe, et al (R124H; doi.org/10.1371/journal.pone.0133397) and Kitamoto et al. (Nature, Scientific Reports, 10, Article No. 2000, 2020), incorporated by reference in their entirety. In some embodiments, the first strand comprises a sequence that is identical to the sequence of SEQ ID NO: 1-19, except for one base that is mismatched to a juxtaposed base in the second strand. In some embodiments, the first strand comprises a sequence that is identical to the sequence of SEQ ID NO: 4, 9 or 11, except for one base that is mismatched to a juxtaposed base in the second strand. In additional embodiments, the mismatched base is between three, four, five, six, or seven bases away from the TGFBI R124H mutation site. In further embodiments, the first strand and the second strand are each between 16 and 23 bases in length. In yet further embodiments, the RNA complex has deoxythymidine overhangs.

In some embodiments, the strand has two or more nucleotides that mismatch the target sequence. In some embodiments, the two or more nucleotides that mismatch the target sequence are positioned consecutively. In some embodiments, the two or more nucleotides that mismatch the target sequence are positioned separately from one another.

In some embodiments, the percentage identity is determined including any overhangs to the strand sequence (e.g., when double uridine overhang is added to a 19-mer sequence, the percentage identity is determined based on the sequence of the 21-mer including the overhang). In some embodiments, the percentage identity is determined excluding any overhangs to the strand sequence (e.g., when double uridine overhang is added to a 19-mer sequence, the percentage identity is determined based on the sequence of the 19-mer, not including the overhang).

In some embodiments, the strand includes a sequence having at least 80% identity to SEQ ID NO: 4.

In some embodiments, the strand includes at least one of: an overhang at the 5′ end of the sequence having at least 80% identity to SEQ ID NO: 4 or an overhang at the 3′ end of the sequence having at least 80% identity to SEQ ID NO: 4. In some embodiments, the strand includes at least one of: cytidine monophosphate (rC) at the 5′ end of the sequence having at least 80% identity to SEQ ID NO: 4, uridine monophosphate-cytidine monophosphate (rU-rC) at the 5′ end of the sequence having at least 80% identity to SEQ ID NO: 4, double uridine monophosphate (rU-rU) at the 3′ end of the sequence having at least 80% identity to SEQ ID NO: 4, double deoxythymidine monophosphates (dT-dT) at the 3′ end of the sequence having at least 80% identity to SEQ ID NO: 4, double 2′-O-methylated guanosine monophosphates (oG-oG) at the 3′ end of the sequence having at least 80% identity to SEQ ID NO: 4, and double 2′-O-methyl-uridine monophosphates (oU-oU) at the 3′ end of the sequence having at least 80% identity to SEQ ID NO: 4.

In some embodiments, the RNA complex further includes a strand (e.g., an antisense strand) that includes a sequence having at least 80% identity to SEQ ID NO: 23. In some embodiments, the sequence having at least 80% identity to SEQ ID NO: 4 and the sequence having at least 80% identity to SEQ ID NO: 23 are located on a same strand (e.g., a single strand that forms a hairpin structure). In some embodiments, the sequence having at least 80% identity to SEQ ID NO: 4 and the sequence having at least 80% identity to SEQ ID NO: 23 are located on separate strands. In some embodiments, the RNA complex further includes a strand that includes a sequence of SEQ ID NO: 23. In some embodiments, the RNA complex further includes a strand that consists of a sequence of SEQ ID NO: 23.

In some embodiments, the strand includes a sequence of SEQ ID NO: 4.

In some embodiments, the strand includes at least one of: an overhang at the 5′ end of the sequence of SEQ ID NO: 4 or an overhang at the 3′ end of the sequence of SEQ ID NO: 4. In some embodiments, the strand includes at least one of: cytidine monophosphate (rC) at the 5′ end of the sequence of SEQ ID NO: 4, uridine monophosphate-cytidine monophosphate (rU-rC) at the 5′ end of the sequence of SEQ ID NO: 4, double uridine monophosphate (rU-rU) at the 3′ end of the sequence of SEQ ID NO: 4, double deoxythymidine monophosphates (dT-dT) at the 3′ end of the sequence of SEQ ID NO: 4, double 2′-O-methylated guanosine monophosphates (oG-oG) at the 3′ end of the sequence of SEQ ID NO: 4, and double 2′-O-methyl-uridine monophosphates (oU-oU) at the 3′ end of the sequence of SEQ ID NO: 4.

In some embodiments, the RNA complex further includes a strand (e.g., an antisense strand) that includes a sequence having at least 80% identity to SEQ ID NO: 23. In some embodiments, the RNA complex further includes a strand that includes a sequence of SEQ ID NO: 23. In some embodiments, the RNA complex further includes a strand that consists of a sequence of SEQ ID NO: 23.

In some embodiments, the strand consists of a sequence of SEQ ID NO: 4.

In some embodiments, the RNA complex further includes a strand (e.g., an antisense strand) that includes a sequence having at least 80% identity to SEQ ID NO: 23. In some embodiments, the RNA complex further includes a strand that includes a sequence of SEQ ID NO: 23. In some embodiments, the RNA complex further includes a strand that consists of a sequence of SEQ ID NO: 23.

In some embodiments, the strand includes a sequence having at least 80% identity to SEQ ID NO: 11.

In some embodiments, the strand includes at least one of: an overhang at the 5′ end of the sequence having at least 80% identity to SEQ ID NO: 11 or an overhang at the 3′ end of the sequence having at least 80% identity to SEQ ID NO: 11.

In some embodiments, the strand includes at least one of: adenosine monophosphate (rA) at the 5′ end of the sequence having at least 80% identity to SEQ ID NO: 11, uridine monophosphate-adenosine monophosphate (rU-rA) at the 5′ end of the sequence having at least 80% identity to SEQ ID NO: 11, double uridine monophosphate (rU-rU) at the 3′ end of the sequence having at least 80% identity to SEQ ID NO: 11, double deoxythymidine monophosphates (dT-dT) at the 3′ end of the sequence having at least 80% identity to SEQ ID NO: 11, 2′-O-methylated cytidine monophosphate-uridine monophosphate (oC-oU) at the 3′ end of the sequence having at least 80% identity to SEQ ID NO: 11, and double 2′-O-methyl-uridine monophosphates (oU-oU) at the 3′ end of the sequence having at least 80% identity to SEQ ID NO: 11.

In some embodiments, the RNA complex further includes a strand (e.g., an antisense strand) that includes a sequence having at least 80% identity to SEQ ID NO: 30. In some embodiments, the sequence having at least 80% identity to SEQ ID NO: 11 and the sequence having at least 80% identity to SEQ ID NO: 30 are located on a same strand (e.g., a single strand that forms a hairpin structure). In some embodiments, the sequence having at least 80% identity to SEQ ID NO: 11 and the sequence having at least 80% identity to SEQ ID NO: 30 are located on separate strands. In some embodiments, the RNA complex further includes a strand that includes a sequence of SEQ ID NO: 30. In some embodiments, the RNA complex further includes a strand that consists of a sequence of SEQ ID NO: 30.

In some embodiments, the strand includes a sequence of SEQ ID NO: 11.

In some embodiments, the strand includes at least one of: an overhang at the 5′ end of the sequence of SEQ ID NO: 11 or an overhang at the 3′ end of the sequence of SEQ ID NO: 11. In some embodiments, the strand includes at least one of: adenosine monophosphate (rA) at the 5′ end of the sequence of SEQ ID NO: 11, uridine monophosphate-adenosine monophosphate (rU-rA) at the 5′ end of the sequence of SEQ ID NO: 11, double uridine monophosphate (rU-rU) at the 3′ end of the sequence of SEQ ID NO: 11, double deoxythymidine monophosphates (dT-dT) at the 3′ end of the sequence of SEQ ID NO: 11, 2′-O-methylated cytidine monophosphate-uridine monophosphate (oC-oU) at the 3′ end of the sequence of SEQ ID NO: 11, and double 2′-O-methyl-uridine monophosphates (oU-oU) at the 3′ end of the sequence of SEQ ID NO: 11.

In some embodiments, the RNA complex further includes a strand that includes a sequence having at least 80% identity to SEQ ID NO: 30. In some embodiments, the RNA complex further includes a strand that includes a sequence of SEQ ID NO: 30. In some embodiments, the RNA complex further includes a strand that consists of a sequence of SEQ ID NO: 30.

In some embodiments, the strand consists of a sequence of SEQ ID NO: 11.

In some embodiments, the RNA complex further includes a strand (e.g., an antisense strand) that includes a sequence having at least 80% identity to SEQ ID NO: 30. In some embodiments, the RNA complex further includes a strand that includes a sequence of SEQ ID NO: 30. In some embodiments, the RNA complex further includes a strand that consists of a sequence of SEQ ID NO: 30.

In some embodiments, the strand includes a sequence having at least 80% identity to SEQ ID NO: 16.

In some embodiments, the strand includes at least one of: an overhang at the 5′ end of the sequence having at least 80% identity to SEQ ID NO: 16 or an overhang at the 3′ end of the sequence having at least 80% identity to SEQ ID NO: 16. In some embodiments, the strand includes at least one of: guanosine monophosphate (rG) at the 5′ end of the sequence having at least 80% identity to SEQ ID NO: 16, double guanosine monophosphate (rG-rG) at the 5′ end of the sequence having at least 80% identity to SEQ ID NO: 16, double uridine monophosphate (rU-rU) at the 3′ end of the sequence having at least 80% identity to SEQ ID NO: 16, double deoxythymidine monophosphates (dT-dT) at the 3′ end of the sequence having at least 80% identity to SEQ ID NO: 16, 2′-O-methylated guanosine monophosphate-cytidine monophosphate (oG-oC) at the 3′ end of the sequence having at least 80% identity to SEQ ID NO: 16, and double 2′-O-methyl-uridine monophosphates (oU-oU) at the 3′ end of the sequence having at least 80% identity to SEQ ID NO: 16.

In some embodiments, the RNA complex further includes a strand that includes a sequence having at least 80% identity to SEQ ID NO: 35. In some embodiments, the sequence having at least 80% identity to SEQ ID NO: 16 and the sequence having at least 80% identity to SEQ ID NO: 35 are located on a same strand (e.g., a single strand that forms a hairpin structure). In some embodiments, the sequence having at least 80% identity to SEQ ID NO: 16 and the sequence having at least 80% identity to SEQ ID NO: 35 are located on separate strands. In some embodiments, the RNA complex further includes a strand that includes a sequence of SEQ ID NO: 35. In some embodiments, the RNA complex further includes a strand that consists of a sequence of SEQ ID NO: 35.

In some embodiments, the strand includes a sequence of SEQ ID NO: 16.

In some embodiments, the strand includes at least one of: an overhang at the 5′ end of the sequence of SEQ ID NO: 16 or an overhang at the 3′ end of the sequence of SEQ ID NO: 16. In some embodiments, the strand includes at least one of: guanosine monophosphate (rG) at the 5′ end of the sequence of SEQ ID NO: 16, double guanosine monophosphate (rG-rG) at the 5′ end of the sequence of SEQ ID NO: 16, double uridine monophosphate (rU-rU) at the 3′ end of the sequence of SEQ ID NO: 16, double deoxythymidine monophosphates (dT-dT) at the 3′ end of the sequence of SEQ ID NO: 16, 2′-O-methylated guanosine monophosphate-cytidine monophosphate (oG-oC) at the 3′ end of the sequence of SEQ ID NO: 16, and double 2′-O-methyl-uridine monophosphates (oU-oU) at the 3′ end of the sequence of SEQ ID NO: 16.

In some embodiments, the RNA complex further includes a strand that includes a sequence having at least 80% identity to SEQ ID NO: 35. In some embodiments, the RNA complex further includes a strand that includes a sequence of SEQ ID NO: 35.

In some embodiments, the RNA complex further includes a strand that consists of a sequence of SEQ ID NO: 35. In some embodiments, the strand consists of a sequence of SEQ ID NO: 16.

In some embodiments, the RNA complex further includes a strand (e.g., an antisense strand) that includes a sequence having at least 80% identity to SEQ ID NO: 35. In some embodiments, the RNA complex further includes a strand that includes a sequence of SEQ ID NO: 35. In some embodiments, the RNA complex further includes a strand that consists of a sequence of SEQ ID NO: 35.

In some embodiments, the strand includes a sequence having at least 80% identity to SEQ ID NO: 9.

In some embodiments, the strand includes at least one of: an overhang at the 5′ end of the sequence having at least 80% identity to SEQ ID NO: 9 or an overhang at the 3′ end of the sequence having at least 80% identity to SEQ ID NO: 9.

In some embodiments, the strand includes at least one of: guanosine monophosphate (rG) at the 5′ end of the sequence having at least 80% identity to SEQ ID NO: 9, uridine monophosphate-guanosine monophosphate (rU-rG) at the 5′ end of the sequence having at least 80% identity to SEQ ID NO: 9, double uridine monophosphate (rU-rU) at the 3′ end of the sequence having at least 80% identity to SEQ ID NO: 9, double deoxythymidine monophosphates (dT-dT) at the 3′ end of the sequence having at least 80% identity to SEQ ID NO: 9, 2′-O-methylated adenosine monophosphate-guanosine monophosphate (oA-oG) at the 3′ end of the sequence having at least 80% identity to SEQ ID NO: 9, and double 2′-O-methyl-uridine monophosphates (oU-oU) at the 3′ end of the sequence having at least 80% identity to SEQ ID NO: 9.

In some embodiments, the RNA complex further includes a strand that includes a sequence having at least 80% identity to SEQ ID NO: 25. In some embodiments, the RNA complex further includes a strand that includes a sequence of SEQ ID NO: 25. In some embodiments, the RNA complex further includes a strand that consists of a sequence of SEQ ID NO: 25.

In some embodiments, the strand includes a sequence of SEQ ID NO: 9.

In some embodiments, the strand includes at least one of: an overhang at the 5′ end of the sequence of SEQ ID NO: 9 or an overhang at the 3′ end of the sequence of SEQ ID NO: 9.

In some embodiments, the strand includes at least one of: guanosine monophosphate (rG) at the 5′ end of the sequence having at least 80% identity to SEQ ID NO: 9, uridine monophosphate-guanosine monophosphate (rU-rG) at the 5′ end of the sequence having at least 80% identity to SEQ ID NO: 9, double uridine monophosphate (rU-rU) at the 3′ end of the sequence having at least 80% identity to SEQ ID NO: 9, double deoxythymidine monophosphates (dT-dT) at the 3′ end of the sequence having at least 80% identity to SEQ ID NO: 9, 2′-O-methylated adenosine monophosphate-guanosine monophosphate (oA-oG) at the 3′ end of the sequence having at least 80% identity to SEQ ID NO: 9, and double 2′-O-methyl-uridine monophosphates (oU-oU) at the 3′ end of the sequence having at least 80% identity to SEQ ID NO: 9.

In some embodiments, the RNA complex further includes a strand that includes a sequence having at least 80% identity to SEQ ID NO: 25. In some embodiments, the RNA complex further includes a strand that includes a sequence of SEQ ID NO: 25. In some embodiments, the RNA complex further includes a strand that consists of a sequence of SEQ ID NO: 25.

In some embodiments, the strand consists of a sequence of SEQ ID NO: 9.

In some embodiments, the RNA complex further includes a strand that includes a sequence having at least 80% identity to SEQ ID NO: 25. In some embodiments, the RNA complex further includes a strand that includes a sequence of SEQ ID NO: 25. In some embodiments, the RNA complex further includes a strand that consists of a sequence of SEQ ID NO: 25.

In accordance with some embodiments, a ribonucleic acid (RNA) complex includes a strand (e.g., an antisense strand) that includes a sequence having at least 80% identity to one of SEQ ID NOs: 20-38.

In some embodiments, the strand includes a sequence having at least 80% identity to SEQ ID NO: 23. In some embodiments, the strand includes at least one of: an overhang at the 5′ end of the sequence having at least 80% identity to SEQ ID NO: 23 or an overhang at the 3′ end of the sequence having at least 80% identity to SEQ ID NO: 23.

In some embodiments, the strand includes a sequence having at least 80% identity to SEQ ID NO: 30. In some embodiments, the strand includes at least one of: an overhang at the 5′ end of the sequence having at least 80% identity to SEQ ID NO: 30 or an overhang at the 3′ end of the sequence having at least 80% identity to SEQ ID NO: 30.

In some embodiments, the strand includes a sequence having at least 80% identity to SEQ ID NO: 35. In some embodiments, the strand includes at least one of: an overhang at the 5′ end of the sequence having at least 80% identity to SEQ ID NO: 35 or an overhang at the 3′ end of the sequence having at least 80% identity to SEQ ID NO: 35.

In accordance with some embodiments, a ribonucleic acid (RNA) complex includes a strand having a sequence overlapping with a sequence of a transforming growth factor beta induced (TGFBI) protein messenger RNA (mRNA), the strand containing adenine at a position corresponding to the c. 371G>A single nucleotide polymorphism (SNP) in exon 4 of the TGFBI gene.

In some embodiments, the RNA complex includes a short interfering RNA duplex. In some embodiments, the RNA complex is a short interfering RNA duplex (e.g., the RNA complex has a double-stranded RNA structure with a sense strand that is shorter than 30-mers and an antisense strand that is shorter than 30-mers). In some embodiments, the RNA complex has a double-stranded RNA structure with a sense strand that is shorter than 24-mers and an antisense strand that is shorter than 24-mers). In some embodiments, the RNA complex has a double-stranded RNA structure with a sense strand that is longer than 18-mers and an antisense strand that is longer than 18-mers).

In some embodiments, the RNA complex includes a double stranded RNA complex configured for forming a short interfering RNA duplex (e.g., the RNA complex has a double-stranded RNA structure with at least one strand longer than 30-mers). In some embodiments, the RNA complex has a double-stranded RNA structure with both strands longer than 30-mers, and in some cases, longer than 50-mers or 100-mers.

In some embodiments, the RNA complex includes an RNA hairpin. For example, the RNA complex may be formed by a single strand containing the sequence for the sense strand and the sequence of the antisense strand on the same single strand.

As explained above, a dicer enzyme located within cells may cleave the double stranded RNA complex or the RNA hairpin to provide siRNA duplexes.

In accordance with some embodiments, a method of preventing, ameliorating, or treating granular corneal dystrophy type 2 in a subject includes administering to the subject any RNA complex described herein. For example, the RNA complex may be delivered into cells by a transfection agent, such as lipofectamine, calcium phosphate, or cationic lipid. Alternatively, the RNA complex may be delivered into cells using electroporation. In some cases, the RNA complex is delivered by viral infection (e.g., using adenovirus, retrovirus, or other viral vectors). In some other cases, nanoparticles may be used to deliver the RNA complexes.

In some embodiments, the administering includes injecting the RNA complex into the subject. For example, a solution containing the RNA complexes is provided by intrastromal injection.

In some embodiments, the administering includes applying a solution containing the RNA complex onto the subject. For example, eye drops containing the RNA complexes may be applied to the eye so that the RNA complexes are absorbed into the eye.

In some embodiments, the administering includes introducing the RNA complex into a cell containing and expressing a deoxyribonucleic acid (DNA) molecule having the c. 371G>A single nucleotide polymorphism (SNP) in exon 4 of a transforming growth factor beta induced (TGFBI) gene. For example, the cell may contain a mutant allele that would produce mutant TGFBI proteins, which will cause GCD2.

In some embodiments, the subject is a vertebrate. In some embodiments, the subject is human.

In some embodiments, the method also includes, prior to administering to the subject the RNA complex: obtaining sequence information of the subject; and determining that the subject has an allele having the c. 371G>A SNP in exon 4 of the TGFBI gene and an allele that does not have the c. 371G>A SNP in exon 4 of the TGFBI gene. In this case, the diagnostic information that the subject has the mutant allele for GCD2 can avoid or reducing treating patients who do not have the mutant allele for GCD2 with the RNA complex described herein. In addition, the diagnostic information that the subject also has the wild type allele indicates that the subject is less likely to experience any adverse effect associated with completely silencing the TGFBI gene (e.g., silencing both alleles), in which case no TGFBI proteins are produced.

In some embodiments, the sequence information of the subject consists of sequence information of exon 4 of the TGFBI gene.

In some embodiments, the sequence information of the subject includes sequence information of a subset, less than all, of exon 4 of the TGFBI gene.

In some embodiments, the sequence information of the subject includes only the sequence information of the c. 371G>A SNP in exon 4 of the TGFBI gene. In some embodiments, the sequence information of the c. 371G>A SNP in exon 4 of the TGFBI gene is obtained by methods that detect a point mutation, such as polymerase chain reaction (PCR) assays (e.g., real-time PCR assays).

In some embodiments, the sequence information of the subject includes whole-genome sequence information of the subject.

### EXAMPLES

### Example 1: siRNA Design

### Methods and Materials

A total of 19 siRNAs were synthesized to screen all possible sequences containing the R124H mutation (Eurofins MWG Operon, Ebersberg, Germany). Each siRNA consisted of a 19-nucleotide with two 3′ deoxythymidine nucleotide overhangs. As controls, a nonspecific siRNA and a luciferase siRNA were also designed, acting to have no specific effect and to inhibit the expression of luciferase, respectively.

| TGFBI 124C WT | 5′-CTCAGCTGTACACGGACCGC |
|---|---|
| | ACGGAGAAGCTGAGGCC-3′ |
| TGFBI 124H | 5′-CTCAGCTGTACACGGACCAC |
| Mutant | ACGGAGAAGCTGAGGCC-3′ |
| 1 | cucagcuguacacggaccaTT |
| 2 | ucagcuguacacggaccacTT |
| 3 | cagcuguacacggaccacaTT |
| 4 | agcuguacacggaccacacTT |
| 5 | gcuguacacggaccacacgTT |
| 6 | cuguacacggaccacacggTT |
| 7 | uguacacggaccacacggaTT |
| 8 | guacacggaccacacggagTT |
| 9 | uacacggaccacacggagaTT |
| 10 | acacggaccacacggagaaTT |
| 11 | cacggaccacacggagaagTT |
| 12 | acggaccacacggagaagcTT |
| 13 | cggaccacacggagaagcuTT |
| 14 | ggaccacacggagaagcugTT |
| 15 | gaccacacggagaagcugaTT |
| 16 | accacacggagaagcugagTT |
| 17 | ccacacggagaagcugaggTT |
| 18 | cacacggagaagcugaggcTT |
| 19 | acacggagaagcugaggccTT |

TABLE 1
siRNA Sense Sequences

| SEQ ID NO | siRNA |
|---|---|
| 1 | cucagcuguacacggacca |
| 2 | ucagcuguacacggaccac |
| 3 | cagcuguacacggaccaca |
| 4 | agcuguacacggaccacac |
| 5 | gcuguacacggaccacacg |
| 6 | cuguacacggaccacacgg |
| 7 | uguacacggaccacacgga |
| 8 | guacacggaccacacggag |
| 9 | uacacggaccacacggaga |
| 10 | acacggaccacacggagaa |
| 11 | cacggaccacacggagaag |
| 12 | acggaccacacggagaagc |
| 13 | cggaccacacggagaagcu |
| 14 | ggaccacacggagaagcug |
| 15 | gaccacacggagaagcuga |
| 16 | accacacggagaagcugag |
| 17 | ccacacggagaagcugagg |
| 18 | cacacggagaagcugaggc |
| 19 | acacggagaagcugaggcc |

TABLE 2
siRNA Antisense Sequences

| SEQ ID NO | siRNA |
|---|---|
| 20 | UGGUCCGUGUACAGCUGAG |
| 21 | GUGGUCCGUGUACAGCUGA |
| 22 | UGUGGUCCGUGUACAGCUG |
| 23 | GUGUGGUCCGUGUACAGCU |
| 24 | CGUGUGGUCCGUGUACAGC |
| 25 | CCGUGUGGUCCGUGUACAG |
| 26 | UCCGUGUGGUCCGUGUACA |
| 27 | CUCCGUGUGGUCCGUGUAC |
| 28 | UCUCCGUGUGGUCCGUGUA |
| 29 | UUCUCCGUGUGGUCCGUGU |
| 30 | CUUCUCCGUGUGGUCCGUG |
| 31 | GCUUCUCCGUGUGGUCCGU |
| 32 | AGCUUCUCCGUGUGGUCCG |
| 33 | CAGCUUCUCCGUGUGGUCC |
| 34 | UCAGCUUCUCCGUGUGGUC |
| 35 | CUCAGCUUCUCCGUGUGGU |
| 36 | CCUCAGCUUCUCCGUGUGG |
| 37 | GCCUCAGCUUCUCCGUGUG |
| 38 | GGCCUCAGCUUCUCCGUGU |

The sense sequence of NSC4 is 5′-UAGCGACUAAACACAUCAAUU-3′ (SEQ ID NO: 39, inverted β-galactosidase sequence with two uracil overhang) and the sense strand of siLUC is 5′-GUGCGUUGCUAGUAC CAACUU-3′ (SEQ ID NO: 40 with two uracil overhang) (both synthesized by Eurofins MWG Operon). The antisense sequence for NSC4 (including SEQ ID NO: 41) and the antisense sequence for siLUC (including SEQ ID NO: 42) are also shown in Table 3.

TABLE 3
Antisense Sequences

| SEQ ID NO | SIRNA |
|---|---|
| 39 | UAGCGACUAAACACAUCAA |
| 40 | GUGCGUUGCUAGUACCAAC |
| 41 | UUGAUGUGUUUAGUCGCUA |
| 42 | GUUGGUACUAGCAACGCAC |

### Cell Culture

AD293 human embryonic kidney cells (Life Technologies) were cultured in Dulbecco's modified Eagle's medium (DMEM)(Invitrogen, Paisley, UK) supplemented with 10% fetal bovine serum (Invitrogen).

### Dual Luciferase Reporter Assay

For siRNA screening, AD293 cells were seeded at 6.5×10³ cells per well in a 96-well plate 24 hours before transfection. Cells were transfected using Lipofectamine 2000 (Invitrogen) according to the manufacturer's instructions. Cells were transfected in quadruplicates with mutated and wild type TGFBI and firefly luciferase, Renilla luciferase expression construct, and the mutation-specific siRNAs at a concentration of 0-6.25 nM; all were diluted in OptiMEM (Invitrogen). A nonspecific control siRNA (NSC4) and a siLUC control, which targets the luciferase component of the vector, were also transfected at the same concentrations as the mutation-specific siRNAs.

The Dual-Luciferase Reporter Assay (Promega, Southampton, UK) was used to measure the effect of siRNA on luciferase expression 24 hours after transfection. The assay was used according to the manufacturer's instructions; the medium was removed and cells were washed with PBS before replacement with passive lysis buffer (Promega). Cells were shaken on a plate shaker for 15 minutes to ensure they were fully lysed, before the activities of both firefly and Renilla luciferase were measured sequentially using the LUMIstar OPTIMA (BMG Labtech, Aylesbury, UK).

### Results

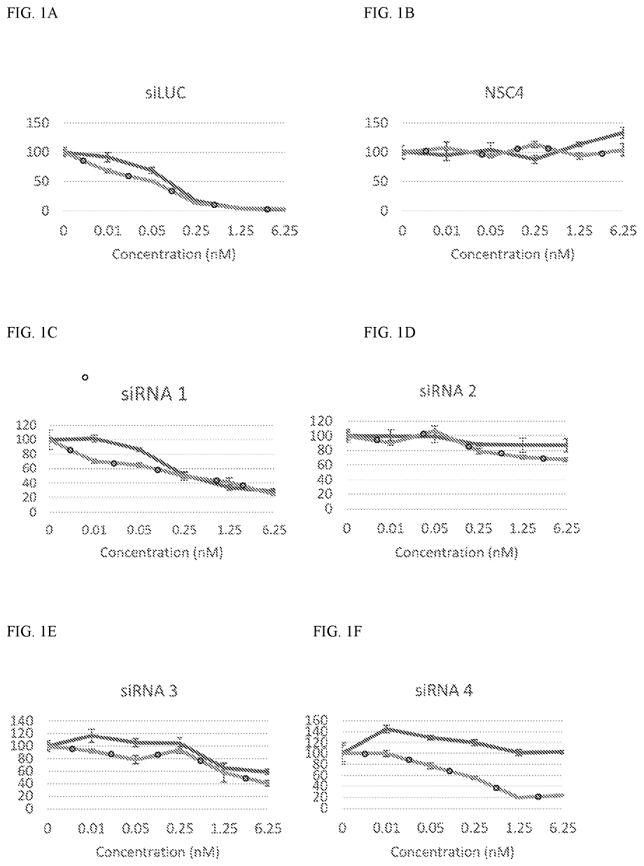

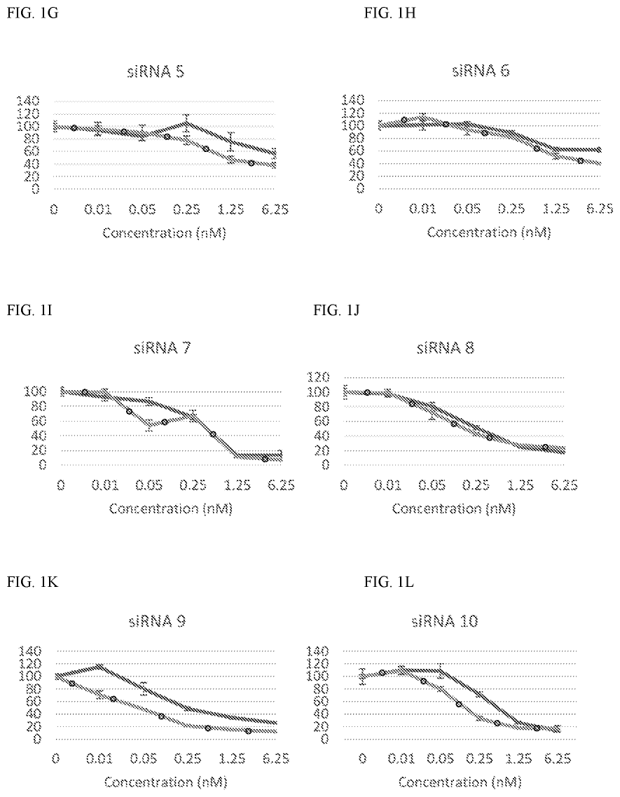

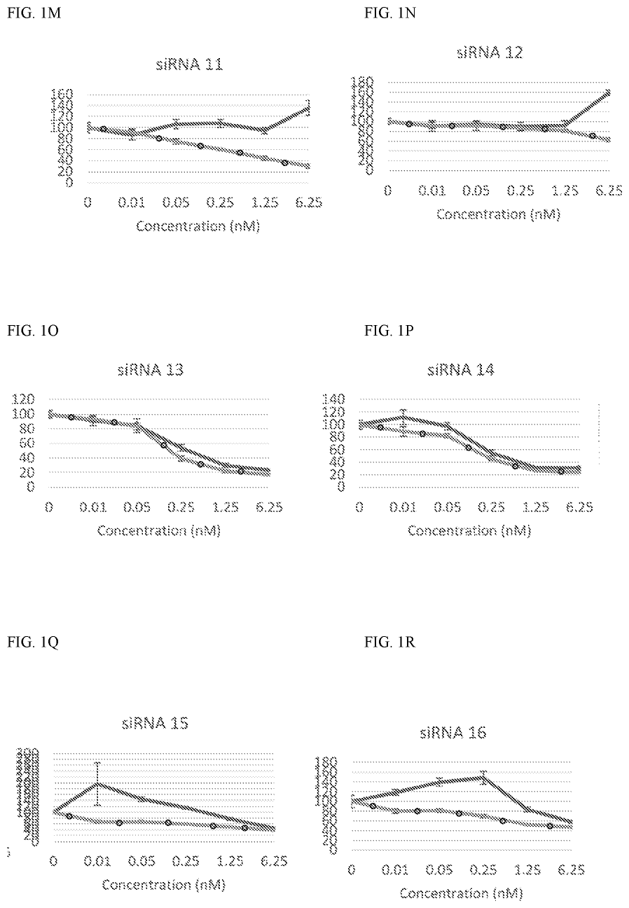

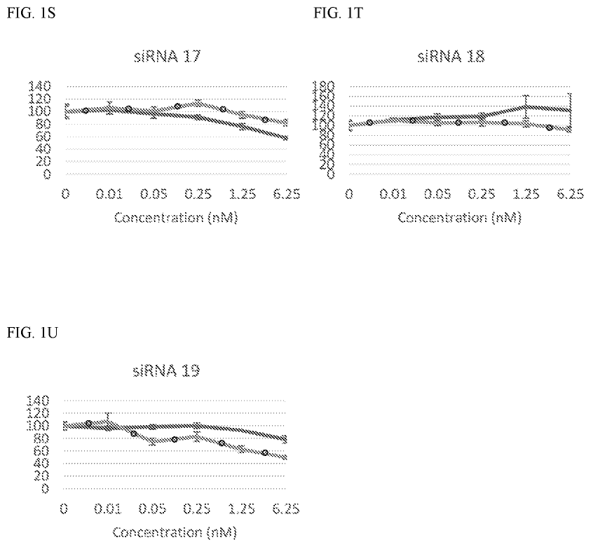

FIG. 1 shows the activity of Luciferase for various constructs. Results shown in each panel are based on an average of eight wells, which correspond to quadruplicate replicates run on two separate occasions. The results for siRNA1 were obtained using SEQ ID NO: 1, the results for siRNA2 were obtained using SEQ ID NO: 2, the results for siRNA3 were obtained using SEQ ID NO: 3, the results for siRNA4 were obtained using SEQ ID NO: 4, the results for siRNA5 were obtained using SEQ ID NO: 5, the results for siRNA6 were obtained using SEQ ID NO: 6, the results for siRNA7 were obtained using SEQ ID NO: 7, the results for siRNA8 were obtained using SEQ ID NO: 8, the results for siRNA9 were obtained using SEQ ID NO: 9, the results for siRNA10 were obtained using SEQ ID NO: 10, the results for siRNA11 were obtained using SEQ ID NO: 11, the results for siRNA12 were obtained using SEQ ID NO: 12, the results for siRNA13 were obtained using SEQ ID NO: 13, the results for siRNA14 were obtained using SEQ ID NO: 14, the results for siRNA15 were obtained using SEQ ID NO: 15, the results for siRNA16 were obtained using SEQ ID NO: 16, the results for siRNA17 were obtained using SEQ ID NO: 17, the results for siRNA18 were obtained using SEQ ID NO: 18, and the results for siRNA19 were obtained using SEQ ID NO: 19.

FIG. 1 shows that SEQ ID NOs: 4, 11, and 16 suppress the expression of the mutant allele (MUT) while maintaining the expression of the wild type allele (WT). Thus, in some embodiments, SEQ ID NO: 4 is used to suppress the expression of the mutant allele. In some embodiments, SEQ ID NO: 11 is used to suppress the expression of the mutant allele. In some embodiments, SEQ ID NO: 16 is used to suppress the expression of the mutant allele. FIG. 1 also shows that SEQ ID NO: 9 has a strong knockdown effect. Thus, in some embodiments, SEQ ID NO: 9 is used to suppress the expression of the mutant allele.

### Example 2: Improved Allele Specificity by an Additional Single Nucleotide Mismatch Adjacent to the R124H Mutation Site and Improved Potency by Modifying Length of Candidate siRNA

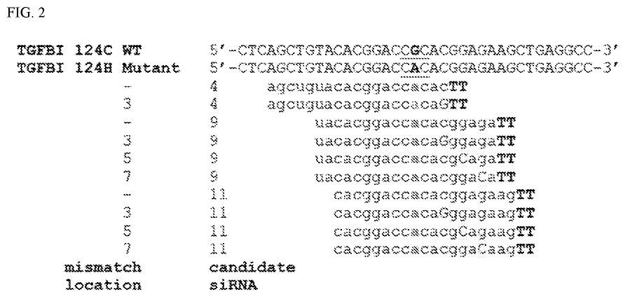

In order to improve the discriminatory power of TGFBI R124H siRNA, new mismatch siRNAs were designed by modifying best two candidates, siRNA 4 and siRNA 11, with high allele-specificity and activity (knockdown of mutant allele), and another one candidate that had overall best potency but lacked strong allele-specificity (siRNA 9) as previously discovered in the gene walk study incorporating all 19 possible sequences, containing the R124H mutation. The mismatch siRNAs contained an additional mismatched nucleotide (incorrectly paired) at 3- and 5-7-bp away from the R124H mutation site and within the seed region of the siRNA (FIG. 2). All siRNAs consisted of a 19-nucleotide duplex with deoxythymidine overhangs (dT-dT) (Eurofins MWG Operon, Ebersberg, Germany).

Additionally, a pool of 3 best candidate siRNAs (siRNA 4, siRNA 11 and siRNA 16) were mixed and transfected together to identify if there was any improvement in overall knockdown of mutant-type plasmid. Prior data suggested that a mixture of siRNA sequences targeting multiple regions within the transcript could be beneficial in reduction of off-target effects. Lastly, the best candidate siRNA was re-designed to include modified lengths varying from 16-bp to 23-bp as shown in the FIG. 3. Additional nucleotides were added or removed either at 5′ or 3′ ends to identify the potential effect of the length on the effectiveness of the siRNA.

### Methods and Materials

Human AD293 cells were cultured in DMEM (Invitrogen, Paisley, UK) supplemented with 10% fetal bovine serum (Invitrogen). For siRNA screening, AD293 cells were seeded at 6.5×10³ cells per well in a 96-well plate 24 hours before transfection. Cells were transfected using Lipofectamine 2000 reagent (Invitrogen) according to the manufacturer's instructions. Cells were transfected in quadruplicate with TGFBI-luciferase wild type or mutated plasmid, co-transfected with Renilla luciferase expression construct for internal control of cell transfection, and the mutation-specific siRNA tested. The study aimed to identify differences between current strongest candidates and their variants that contained an additional mismatch nucleotide in the sequence. The assay was performed at two doses of each siRNA prepared by dilution in OptiMEM (Invitrogen), and the concentrations used were as follows: 0 nM (untreated), 0.25 nM (low dose) and 6.5 nM (high dose). The same methodology was used to assess the modified lengths of the candidate siRNA.

As controls, nonspecific siRNA (NSC4) and siRNA targeting the luciferase component of the vector (siLUC) were also transfected at the same concentrations as the mutation-specific siRNAs. Control siRNAs were designed to have no specific effect (NSC4) and to inhibit the expression of luciferase reporter incorporated into TGFBI expression construct (siLUC), respectively. The sense sequence of NSC4 is 5′-UAGCGACUAAACACAUCAAUU-3′ (inverted β-galactosidase sequence) and the sense strand of siLUC is 5′-GUGCGUUGCUAGUACCAACUU-3′ (both synthesized by Eurofins MWG Operon).

The Dual-Luciferase Reporter Assay (Promega, Southampton, UK) was used to measure the effect of siRNA on TGFBI-luciferase expression 24 hours after transfection. The assay was used according to the manufacturer's instructions; briefly, the medium was removed, and cells were washed with PBS before replacement with passive lysis buffer (Promega). Cells were shaken on a plate shaker for 15 minutes to ensure they were fully lysed, before the activities of both firefly and Renilla luciferase were measured sequentially using the LUMIstar OPTIMA (BMG Labtech, Aylesbury, UK). The results were normalised to the untreated wells (0 nM siRNA) and an average knockdown was calculated with standard error bars included.

Results for siRNA with Mismatched Nucleotide

It was confirmed that unmodified siRNA 4, siRNA 9 and siRNA 11 showed high mutant allele knockdown at either low or high dose (Table 4). Pooled siRNA sample did not have a positive effect on TGFBI R124H silencing, suggesting that the use of individual siRNA sequences would yield better results, due to the very strict design requirements for allele specific targeting.

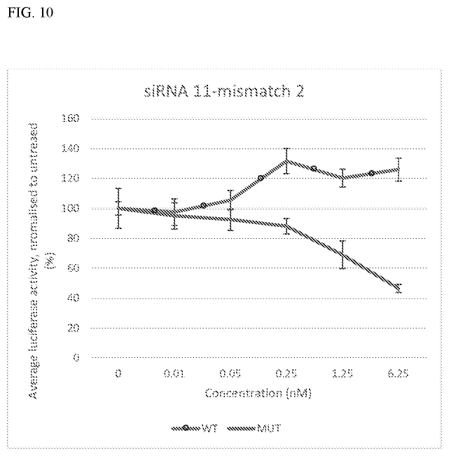

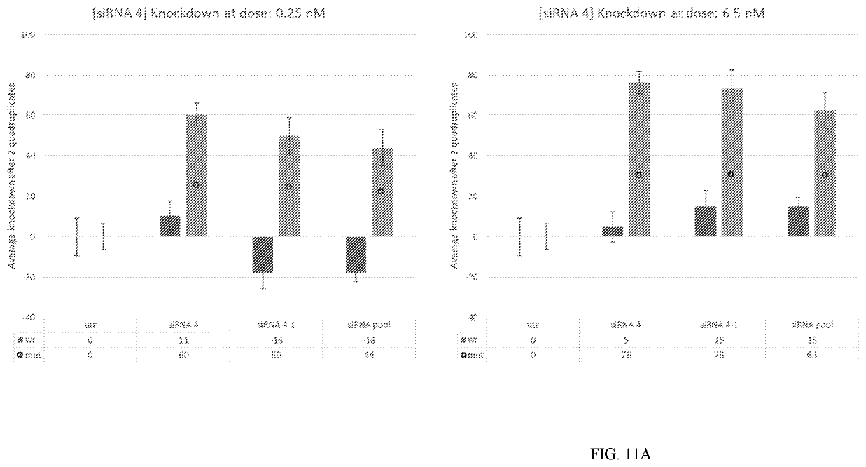

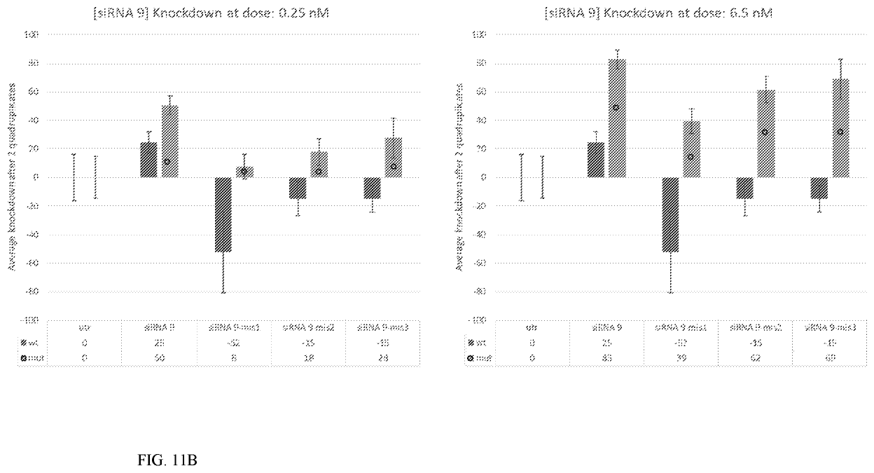

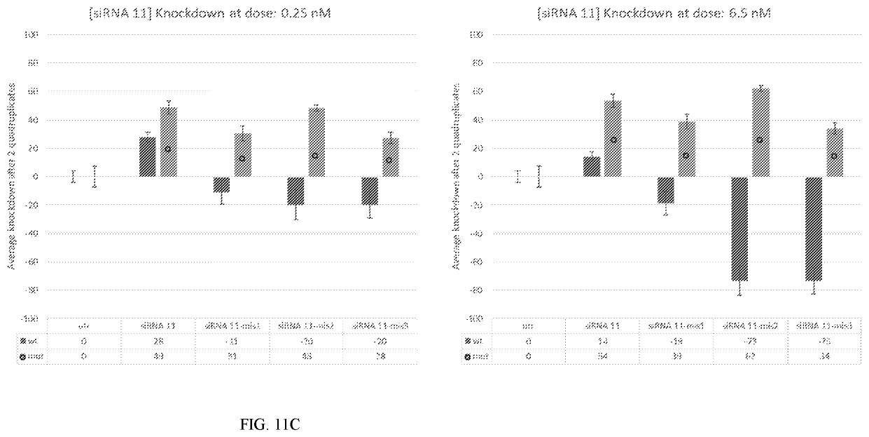

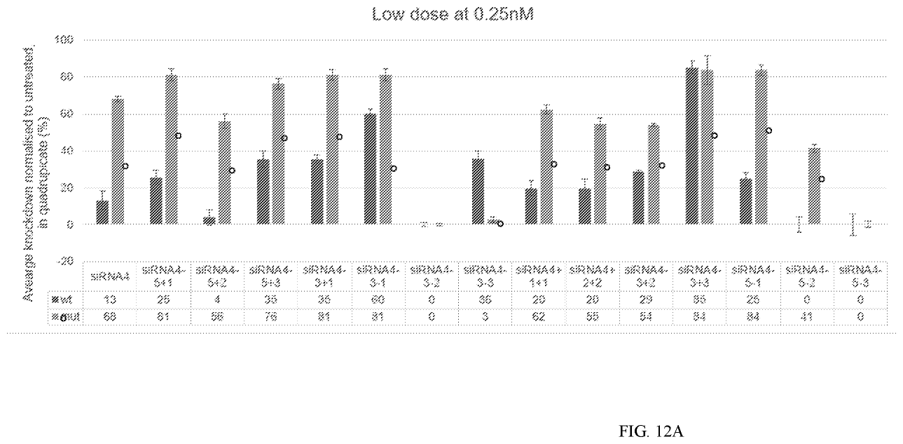

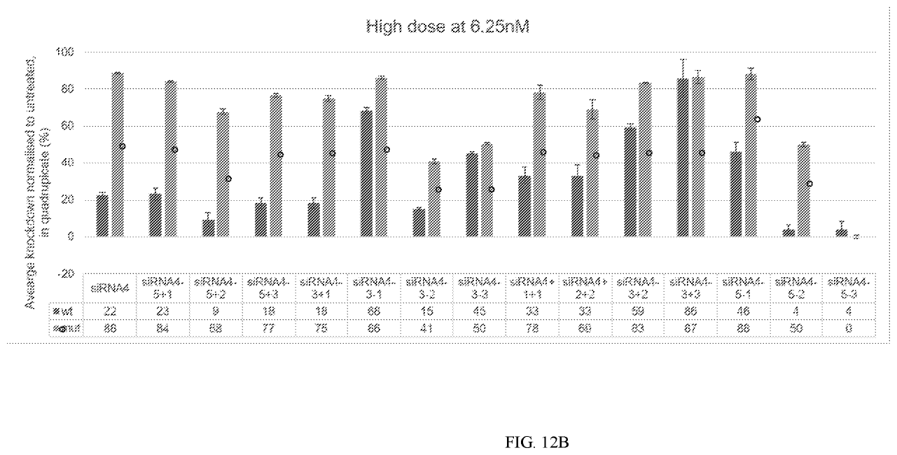

When an additional mismatched nucleotide was introduced, the allele-specificity was improved. In general, it was observed that the mismatch siRNAs had better discriminatory power between wild type and mutant allele. Mismatch versions of siRNA 11 only targeted the mutant allele, with mismatch at 5 bp away from the mutation site (siRNA 11-mismatch 2) being the most potent. All mismatch variants of siRNA 9 showed no or little effect on wild type allele (improved specificity), but reduced knockdown of mutant allele. Introduction of mismatch nucleotide into siRNA 4 sequence did not show any improvement (siRNA 4-mismatch 1). Therefore, both options (siRNA 4 and siRNA 11-mismatch 2) were studied further, with results presented in FIGS. 10-12 and summarized in Table 4. Table 4 shows average knockdown of all siRNA with mismatch modifications tested with difference between wild type (wt) and mutant (mut) allele knockdown included, with negative knockdown values rounded to 0 to calculate differences between wt and mut knockdown.

TABLE 4
Summary of average knockdown of all siRNA with mismatch modifications
Percentage average knockdown (±SE)

| | | | | | | | | | Difference | |
|---|---|---|---|---|---|---|---|---|---|---|
| | | | | | | | | | (wt vs mut) | |
| | low dose (0.25 nM) | | | | high dose (6.25 nM) | | | | low dose | high dose |
| | wt | | mut | | wt | | mut | | (0.25 nM) | (6.25 nM) |
| untreated | 0 | (±9) | 0 | (±6) | 0 | (±15) | 0 | (±7) | 0 | 0 |
| NSC control | 0 | (±8) | 16 | (±14) | 2 | (±5) | 0 | (±9) | 16 | −3 |
| siLuc control | 56 | (±7) | 55 | (±8) | 91 | (±1) | 89 | (±2) | −1 | −3 |
| siRNA4 | 10 | (±7) | 60 | (±6) | 4 | (±13) | 76 | (±3) | 50 | 71 |
| siRNA4-mismatch 1 (at 3 bp) | 0 | (±8) | 50 | (±9) | 14 | (±8) | 73 | (±5) | 50 | 58 |
| siRNA pool (4-11-16) | 0 | (±4) | 44 | (±9) | 15 | (±11) | 62 | (±2) | 44 | 48 |
| siRNA 9 | 25 | (±7) | 50 | (±6) | 61 | (±4) | 83 | (±3) | 26 | 22 |
| siRNA 9-mistmatch 1 (at 3 bp) | 0 | (±28) | 8 | (±8) | 0 | (±14) | 49 | (±6) | 8 | 49 |
| siRNA 9-mistmatch 2 (at 5 bp) | 0 | (±12) | 18 | (±9) | 21 | (±9) | 63 | (±3) | 18 | 42 |
| siRNA 9-mistmatch 3 (at 7 bp) | 0 | (±10) | 28 | (±14) | 21 | (±5) | 72 | (±4) | 28 | 50 |
| siRNA 11 | 28 | (±3) | 49 | (±5) | 14 | (±4) | 55 | (±4) | 21 | 39 |
| siRNA 11-mismatch 1 (at 3 bp) | 0 | (±4) | 30 | (±5) | 0 | (±8) | 39 | (±3) | 31 | 39 |
| siRNA 11-mismatch 2 (at 5 bp) | 0 | (±10) | 48 | (±2) | 0 | (±5) | 62 | (±3) | 48 | 62 |
| siRNA 11-mismatch 3 (at 7 bp) | 0 | (±9) | 28 | (±4) | 0 | (±13) | 34 | (±4) | 28 | 34 |

Based on the results, siRNA 4 was identified as the best candidate due to demonstrating the highest knockdown at low and high doses, as well as the highest discrimination between wild type and mutant type allele, which therefore indicated that siRNA 4 would have the potentially best therapeutic potential.

Results for siRNA4 with Length Modifications

The siRNA4 was re-designed to include modified lengths varying from 16-bp to 23-bp to identify the potential effect of the length on the effectiveness of the siRNA. When the length of the baseline siRNA4 was altered the effect varied significantly. The majority of the siRNAs lost their efficacy or allele-specificity as seen in Table 5, which shows average knockdown of siRNA with length modifications tested with difference between wild type (wt) and mutant (mut) allele knockdown included, where negative knockdown values were rounded to 0 to calculate differences between wt and mut knockdown and highlighted are top 5 siRNA that performed the best. However, some sequences showed similar activity as the original candidate.

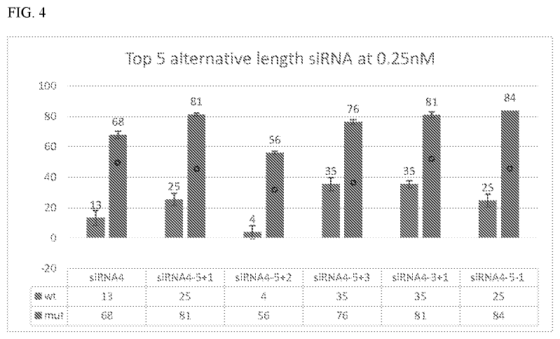

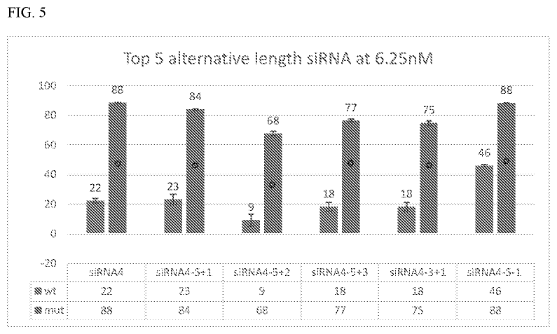

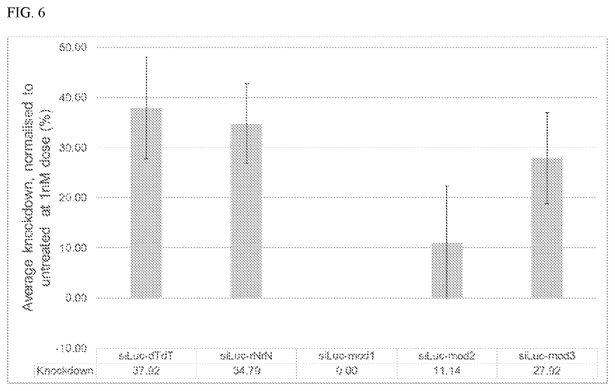

FIGS. 4 and 5 show top 5 best performing siRNA at low (0.25 nM) and high (6.25 nM) doses, which were. siRNA4-5+1, siRNA4-5+2, siRNA4-5+3, siRNA4-3+1, siRNA4-5−1. Out of those siRNA, siRNA4-5+1 demonstrated the best efficacy, where mut was knocked down by 81% at low dose and 84% at high dose, but the allele discrimination was negatively affected because 25% (low dose) and 23% (high dose) of wt was knocked down by the siRNA4-5+1, compared to siRNA4, which showed 13%/22% knockdown of wt. In addition, siRNA4-5−1 demonstrated excellent efficacy at low dose, but once the dose was increased to 6.25 nM, the allele discrimination suffered and 46% of wt was knocked down (FIGS. 10-12).

TABLE 5
Summary of average knockdown of siRNA with length modifications
Percentage average knockdown

| | low dose | | high dose | | Change (wt vs mut) | |
|---|---|---|---|---|---|---|
| | (0.25 nM) | | (6.25 nM) | | low dose | high dose |
| | wt | mut | wt | mut | (0.25 nM) | (6.25 nM) |
| siRNA4 | 13 | 68 | 22 | 88 | 55 | 66 |
| siRNA4-5 + 1 | 25 | 81 | 23 | 84 | 56 | 61 |
| siRNA4-5 + 2 | 4 | 56 | 9 | 68 | 52 | 58 |
| siRNA4-5 + 3 | 35 | 76 | 18 | 77 | 41 | 58 |
| siRNA4-3 + 1 | 35 | 81 | 18 | 75 | 46 | 56 |
| siRNA4-3 + 2 | 29 | 54 | 59 | 83 | 25 | 24 |
| siRNA4-3 + 3 | 85 | 84 | 86 | 87 | −2 | 1 |
| siRNA4-5 − 1 | 25 | 84 | 46 | 88 | 59 | 42 |
| siRNA4-5 − 2 | 0 | 41 | 4 | 50 | 41 | 46 |
| siRNA4-5 − 3 | 0 | 0 | 4 | 0 | 0 | −4 |
| siRNA4-3 − 1 | 60 | 81 | 68 | 86 | 21 | 18 |
| siRNA4-3 − 2 | 0 | 0 | 15 | 41 | 0 | 26 |
| siRNA4-3 − 3 | 36 | 3 | 45 | 50 | −33 | 5 |
| siRNA4 + 1 + 1 | 20 | 62 | 33 | 78 | 43 | 45 |
| siRNA4 + 2 + 2 | 20 | 55 | 33 | 69 | 35 | 36 |

Based on the results, siRNA 4 was identified as the best candidate due to demonstrating the highest knockdown at low and high doses, as well as the highest discrimination between wild type and mutant type allele, which therefore indicated that siRNA 4 would have the potentially best therapeutic potential. The siRNA 11-mismatch 2 was identified as an alternative candidate, because despite demonstrating slightly lower potency than sequence 4, the siRNA did not affect the healthy wild type allele (FIGS. 10-12).

When the length of siRNA4 sequence was altered, a variable effect on its activity and specificity was observed. Although the siRNA4-5+1 offered better potency while the difference between wild-type and mutant type remained similar to siRNA4, an additional length could increase the potential for off-targets that could have a detrimental effect on unintended pathways. The siRNA4-5+1 could be a viable alternative to the siRNA4 candidate if the additional potency were required for in vivo applications. Nonetheless, that would require new designs for the chemical modifications which were already optimised for 19-nt standard siRNA design.

### Example 3: The Effect of Chemical Modification Design on the Activity and Serum Stability of siRNA

An essential aspect of the chemical modifications of siRNAs is to improve their utility in therapeutics by refining their drug-like aspects. Those include the overall stability (resistance to nuclease degradation), duration of gene silencing effect, increased specificity and decreased cellular toxicity. To achieve this improved utility, various modifications could be applied and be experimentally verified in the context of Avellino corneal dystrophy by comparing the effect of chemical modifications on allele-specificity and stability of the siRNA molecule. Three candidates for potential modifications were chosen to further increase efficacy of siRNA candidates derived from the gene walk.

The effect of chemical modification design on the activity and stability of siRNA molecule was investigated. The luciferase-targeting siRNA (siLuc) was chosen because of: (a) the rapid detection of luciferase reporter gene expression, and (b) the possibility of performing both in vivo and in vitro experiments using the same siLuc sequence as the luciferase, which can be expressed in cells via a plasmid and natively in transgenic bioluminescence reporter mice. A literature search was conducted to find chemical modifications that could improve the overall performance of the candidate siRNAs, which were: siLuc-mod1, siLuc-mod2, siLuc-mod3. These were compared against unmodified siRNA sequence that contained either dTdT overhangs or rNrN overhangs. The most effective pattern was applied to the best allele-specific candidate siRNA.

Methods and Materials: Designing Chemically Modified siRNA

siLuc-unmodified. This was a standard design, that contained 19 bp and dTdT overhangs for each strand. Using this siRNA as baseline, three different chemically modified siRNA were compared.

| | |
|---|---|
| sense | 5′- C G A C A A G C C U G G C G C A G U A dT dT -3′ |
| antisense | 3′- dT dT G C U G U U C G G A C C G C G U C A U -5′ |

siLuc-rNrN. This variant of naked 19 bp siRNA with rNrN overhangs was added to examine the resistance to nucleases caused by dTdT overhangs. In the sense strand, GG nucleotides were added as overhangs; whereas, in the anti-sense strand, UC nucleotides were added, both of which matched the luc2 gene sequence.

| | |
|---|---|
| sense | 5′- C G A C A A G C C U G G C G C A G U A G G -3′ |
| antisense | 3′- C U G C U G U U C G G A C C G C G U C A U -5′ |

siLuc-mod1—minimally modified siRNA. The design included: (1) The sense strand having a series of repeating 2′-OMe (a methyl group was added to the 2′ hydroxyl of the ribose moiety of a nucleoside), which inhibits RNAi activity and prevents off-target effects involving sense strand; (2) Additional 2′-OMe added in the guide strand in locations as follows: (a) seed region to reduce off-target effects, and (b) 3′-end to protect the strand from nucleases; and (3) dTdT overhangs included to further protect the siRNA from breakdown by nucleases.

| | | | | | | | | | | | | | | | | | | | | | | | | | |
|---|---|---|---|---|---|---|---|---|---|---|---|---|---|---|---|---|---|---|---|---|---|---|---|---|---|
| sense | 5′- | | | Cₘ | G | A | Cₘ | A | A | G | Cₘ | Cₘ | Uₘ | G | G | Cₘ | G | Cₘ | A | G | Uₘ | A | dT | dT | -3′ |
| antisense | 3′- | dT | dT | G | C | Uₘ | G | U | U | C | G | G | A | C | C | G | Cₘ | G | U | Cₘ | A | Uₘ | | | -5′ |

siLuc-mod 2-fully modified asymmetric siRNA. The design included: (1) the sense strand shortened to 15 bp, which prevents the sense strand from being loaded to RISC and hence prevents all sense strand off-target effects; (2) on the anti-sense strand, the application of an alternating pattern made of 2′-OMe (2′-O-Methyl-ribonucleotide) and 2′-F (2′-deoxy-2′-fluoro-ribonucleotide), where the best pattern would replace the most of pyrimidines 2′-F-RNA; (3) on the sense strand, the application of alternating 2′-OMe and 2′-F pattern but starting with different modification first, such as: (a) MFMFMF<-sense, and (b) FMFMFM<-anti-sense; (4) at 5′ end of antisense strand, restored 5′-Phosphate; and (5) additional phosphorothioate links (*) on the 3′ and 5′ ends of both strands.

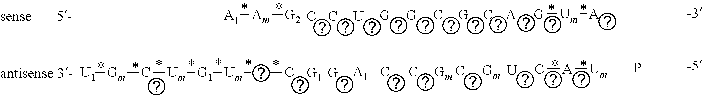

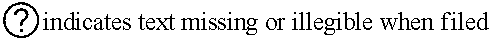

siLuc-mod3—partially modified, based on literature search. The design included: (1) two 2′-OMe units at the 5′-end of the sense strand, which reduces off-target effects by blocking passenger strand and promoting RISC loading of the antisense strand; (2) single 2′-OMe at position 2 from the 5′ end of the guide strand, which improves siRNA specificity, reduces off-target effects related to seed region homology to the 3′ UTR of mRNA (blocks involvement in miRNA pathway); (3) incorporation of at least two 2′-OMe modifications at either U or G residues in the sense strand (other than those at position 9), which reduces immunostimulatory effects; (4) introduction of PS bonds, which improves 3′-exonuclease stability) in antisense dTdT overhang to stabilize it, as opposed to leaving simple dTdT in sense destabilises passenger strand and maintaining immunostimulatory effects; and (5) all pyrimidines in antisense strand replaced with 2′F-RNA units (if includes the first nucleotide at the 5′ end, the 5′P to be restored).

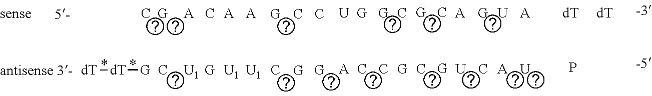

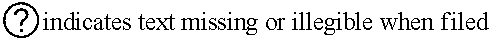

### Dual-Luciferase Reporter Assay

Human AD293 cells were cultured in DMEM (Invitrogen, Paisley, UK) supplemented with 10% foetal bovine serum (Invitrogen). For chemical modification screening, AD293 cells were seeded at 6.5×10³ cells per well in a 96-well plate 24 hours before transfection. Cells were transfected using Lipofectamine 2000 reagent (Invitrogen) according to the manufacturer's instructions. Cells were transfected in quadruplicate with luc2 plasmid, co-transfected with Renilla luciferase expression construct for internal control of cell transfection, and the chemically modified siRNA tested. The experiment aimed to identify any loss of knockdown caused by addition of chemical modifications to the siRNA. The assay was performed at two concentrations: 0 nM (untreated) and 1 nM.

The Dual-Luciferase Reporter Assay (Promega, Southampton, UK) was used to measure the effect of siRNA on luc2 expression 24 hours after transfection. The assay was used according to the manufacturer's instructions; briefly, the medium was removed, and cells were washed with PBS before replacement with passive lysis buffer (Promega). Cells were shaken on a plate shaker for 15 minutes to ensure they were fully lysed, before the activities of both firefly and Renilla luciferase were measured sequentially using the LUMIstar OPTIMA (BMG Labtech, Aylesbury, UK). The results were normalised to the untreated wells (0 nM siRNA) and an average knockdown was calculated with standard error bars included and in addition the results were presented as relative difference in knockdown when compared to unmodified siLuc siRNA.

### Sirna Stability Assay

The siRNA stability assay was used to measure the resistance to nucleases and stability of the siRNA. Stocks containing 4 μg of siRNA (in 20 μL nuclease-free water) were added to foetal bovine serum (Invitrogen) to make up 80% FBS in 100 μl total volume and samples were incubated at 37° C. constant. Timepoints used were as follows: 0, 0.5, 1, 2, 4, 6, 24, 48, 72, and 78 hours. Aliquots of 5 μl were taken at each timepoints (equivalent of 200 ng) and added to 6× loading buffer and subsequently snap frozen on dry ice and stored in −80° C. Aliquots were analyzed by using gel electrophoresis with 2% TBE agarose gels for 20 mins at 100V.

### Results

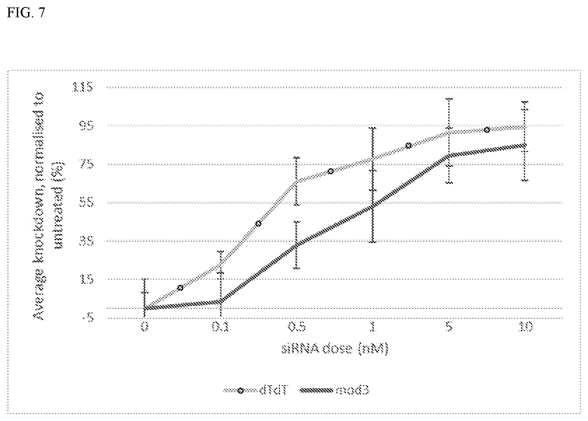

Unmodified siLuc induced a significant knockdown of luciferase expression, regardless of type of 3′ overhangs used (dTdT or nNrN) (FIG. 3). Chemical modifications in siLuc-mod1 deactivated the siRNA and thus no knockdown was observed. Conversely, siLuc-mod2 showed impaired performance, with average knockdown being 27% lower than unmodified siRNA. Finally, siLuc-mod3 showed the best performance out of all chemically modified variants with minimum loss of knockdown being less than 1% and maximum loss of knockdown being 18% with overall 10% lower activity in average when compared to unmodified siLuc. A full titration of siLuc-mod3 and unmodified siLuc-dTdT was also analysed, demonstrating consistent penalty when chemical modification was applied to the sequence which started to overlap at higher than InM doses and at the 0.1 nM dose (FIG. 7). This slight difference in activity could be a result of transfection conditions being fixed to 0.1-10 nM concentration of siRNA, while the molecular weight of each of the sequences was varied (unmodified siLuc-dTdT is 13 345 g/mol and siLuc-mod3 is 13 563 g/mol).

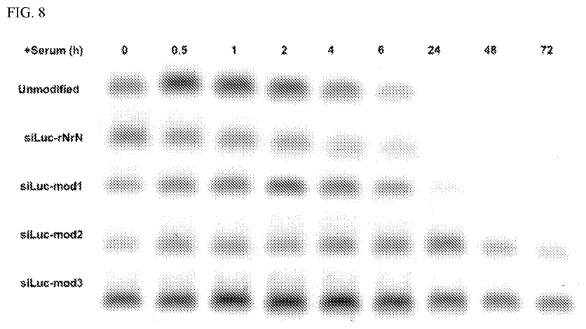

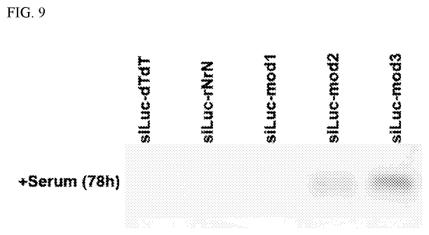

Some chemical modifications were observed to increase stability of the siRNA (FIG. 8). The rNrN almost fully degraded within 6 h, thus showed to be less stable than siRNA with dTdT overhangs and chemically modified variants. Only a small portion of undegraded siRNA with standard design (siLuc-dTdT) was detected after 6 h incubation, but the sequence was fully degraded at 24 h. Partial chemical modification with only 2′OMe had little effect on nuclease resistance, since at 24 h, siLuc2-mod1 exhibited only a small amount of undegraded siRNA remaining. In contrast, fully modified siLuc2-mod2 showed stability from 0 to 24 h, while the amounts started to drop at 48 to 72 h. However, it was posited that this chemical modification caused a considerable, relative loss of knockdown, making it a worse candidate than siLuc-mod3. The best performing variant was siLuc-mod3, which demonstrated strong stability up to 24 h, with a lower amount of siRNA remaining stable siRNA from 48 to 72 h. An additional timepoint of 78 h was visualised on a separate agarose gel, demonstrating that both siLuc-mod2 and siLuc-mod3 showed faint remains of undegraded siRNA with siLuc-mod3 having higher presence, hence showing the best stability (FIG. 9).

Based on the results, the best performing chemical modification was identified to be siLuc-mod3, which showed significantly improved stability with minimal loss of silencing activity caused by introduction of the chemical modifications to the sequence. Thus, in some embodiments, this chemical modification is applied to the candidate TGFBI-R124H siRNA.

### Embodiments

Embodiment 1. A ribonucleic acid (RNA) complex comprising a strand that comprises a sequence having at least 80% identity to one of SEQ ID NOs: 1-19.

Embodiment 2. The RNA complex of any of the above or below embodiments, wherein the strand comprises a sequence having at least 80% identity to SEQ ID NO: 4.

Embodiment 3. The RNA complex of any of the above or below embodiments, wherein the strand includes at least one of: an overhang at the 5′ end of the sequence having at least 80% identity to SEQ ID NO: 4 or an overhang at the 3′ end of the sequence having at least 80% identity to SEQ ID NO: 4.

Embodiment 4. The RNA complex of any of the above or below embodiments, wherein the strand includes at least one of: cytidine monophosphate (rC) at the 5′ end of the sequence having at least 80% identity to SEQ ID NO: 4, uridine monophosphate-cytidine monophosphate (rU-rC) at the 5′ end of the sequence having at least 80% identity to SEQ ID NO: 4, double uridine monophosphate (rU-rU) at the 3′ end of the sequence having at least 80% identity to SEQ ID NO: 4, double deoxythymidine monophosphates (dT-dT) at the 3′ end of the sequence having at least 80% identity to SEQ ID NO: 4, double 2′-O-methylated guanosine monophosphates (oG-oG) at the 3′ end of the sequence having at least 80% identity to SEQ ID NO: 4, and double 2′-O-methyl-uridine monophosphates (oU-oU) at the 3′ end of the sequence having at least 80% identity to SEQ ID NO: 4.

Embodiment 5. The RNA complex of any of the above or below embodiments, wherein the RNA complex further comprises a strand that comprises a sequence having at least 80% identity to SEQ ID NO: 23.

Embodiment 6. The RNA complex of any of the above or below embodiments, wherein the RNA complex further comprises a strand that comprises a sequence of SEQ ID NO: 23.

Embodiment 7. The RNA complex of any of the above or below embodiments, wherein the RNA complex further comprises a strand that consists of a sequence of SEQ ID NO: 23.

Embodiment 8. The RNA complex of any of the above or below embodiments, wherein the strand comprises a sequence of SEQ ID NO: 4.

Embodiment 9. The RNA complex of any of the above or below embodiments, wherein the strand includes at least one of: an overhang at the 5′ end of the sequence of SEQ ID NO: 4 or an overhang at the 3′ end of the sequence of SEQ ID NO: 4.

Embodiment 10. The RNA complex of any of the above or below embodiments, wherein the strand includes at least one of: cytidine monophosphate (rC) at the 5′ end of the sequence of SEQ ID NO: 4, uridine monophosphate-cytidine monophosphate (rU-rC) at the 5′ end of the sequence of SEQ ID NO: 4, double uridine monophosphate (rU-rU) at the 3′ end of the sequence of SEQ ID NO: 4, double deoxythymidine monophosphates (dT-dT) at the 3′ end of the sequence of SEQ ID NO: 4, double 2′-O-methylated guanosine monophosphates (oG-oG) at the 3′ end of the sequence of SEQ ID NO: 4, and double 2′-O-methyl-uridine monophosphates (oU-oU) at the 3′ end of the sequence of SEQ ID NO: 4.

Embodiment 11. The RNA complex of any of the above or below embodiments, wherein the RNA complex comprises a first strand and a second strand, each comprising at least one TGFBI R124H mutation site compared to a wild-type 124C TGFBI gene.

Embodiment 12. The RNA complex of any of the above or below embodiments, wherein the first strand comprises a sequence that is identical to the sequence of SEQ ID NO: 4, except for one base that is mismatched to a juxtaposed base in the second strand.

Embodiment 13. The RNA complex of any of the above or below embodiments, wherein the first strand and the second strand each comprise one TGFBI RI24H mutation site.

Embodiment 14. The RNA complex of any of the above or below embodiments, wherein the mismatched base is between three and seven bases away from the TGFBI R124H mutation site.

Embodiment 15. The RNA complex of any of the above or below embodiments, wherein the mismatched base is three bases away from the TGFBI R124H mutation site.

Embodiment 16. The RNA complex of any of the above or below embodiments, wherein the mismatched base is four bases away from the TGFBI R124H mutation site.

Embodiment 17. The RNA complex of any of the above or below embodiments, wherein the mismatched base is five bases away from the TGFBI R124H mutation site.

Embodiment 18. The RNA complex of any of the above or below embodiments, wherein the mismatched base is six bases away from the TGFBI R124H mutation site.

Embodiment 19. The RNA complex of any of the above or below embodiments, wherein the mismatched base is seven bases away from the TGFBI R124H mutation site.

Embodiment 20. The RNA complex of any of the above or below embodiments, wherein the first strand and the second strand are each between 16 and 23 bases in length.

Embodiment 21. The RNA complex of any of the above or below embodiments, wherein the first strand and the second strand are each 22 bases in length.

Embodiment 22. The RNA complex of any of the above or below embodiments, wherein the RNA complex has deoxythymidine overhangs.

Embodiment 23. The RNA complex of any of the above or below embodiments, wherein the first strand comprises a sequence that is identical to the sequence of SEQ ID NO: 9, except for one base that is mismatched to a juxtaposed base in the second strand.

Embodiment 24. The RNA complex of any of the above or below embodiments, wherein the first strand and the second strand each comprise one TGFBI R124H mutation site.

Embodiment 25. The RNA complex of any of the above or below embodiments, wherein the mismatched base is between three and seven bases away from the TGFBI R124H mutation site.

Embodiment 26. The RNA complex of any of the above or below embodiments, wherein the mismatched base is three bases away from the TGFBI R124H mutation site.

Embodiment 27. The RNA complex of any of the above or below embodiments, wherein the mismatched base is four bases away from the R124H mutation site.

Embodiment 28. The RNA complex of any of the above or below embodiments, wherein the mismatched base is five bases away from the R124H mutation site.

Embodiment 29. The RNA complex of any of the above or below embodiments, wherein the mismatched base is six bases away from the R124H mutation site.

Embodiment 30. The RNA complex of any of the above or below embodiments, wherein the mismatched base is seven bases away from the R124H mutation site.

Embodiment 31. The RNA complex of any of the above or below embodiments, wherein the first strand and the second strand are each between 16 and 23 bases in length.

Embodiment 32. The RNA complex of any of the above or below embodiments, wherein the first strand and the second strand are each 22 bases in length.

Embodiment 33. The RNA complex of any of the above or below embodiments, wherein the RNA complex has deoxythymidine overhangs.

Embodiment 34. The RNA complex of any of the above or below embodiments, wherein the first strand comprises a sequence that is identical to the sequence of SEQ ID NO: 11, except for one base that is mismatched to a juxtaposed base in the second strand.

Embodiment 35. The RNA complex of any of the above or below embodiments, wherein the first strand and the second strand each comprise one R124H mutation site.

Embodiment 36. The RNA complex of any of the above or below embodiments, wherein the mismatched base is between three and seven bases away from the R124H mutation site.

Embodiment 37. The RNA complex of any of the above or below embodiments, wherein the mismatched base is three bases away from the R124H mutation site.

Embodiment 38. The RNA complex of any of the above or below embodiments, wherein the mismatched base is four bases away from the R124H mutation site.

Embodiment 39. The RNA complex of any of the above or below embodiments, wherein the mismatched base is five bases away from the R124H mutation site.

Embodiment 40. The RNA complex of any of the above or below embodiments, wherein the mismatched base is six bases away from the R124H mutation site.

Embodiment 41. The RNA complex of any of the above or below embodiments, wherein the mismatched base is seven bases away from the R124H mutation site.

Embodiment 42. The RNA complex of any of the above or below embodiments, wherein the first strand and the second strand are each between 16 and 23 bases in length.

Embodiment 43. The RNA complex of any of the above or below embodiments, wherein the first strand and the second strand are each 22 bases in length.

Embodiment 44. The RNA complex of any of the above or below embodiments, wherein the RNA complex has deoxythymidine overhangs.

Embodiment 45. The RNA complex of any of the above or below embodiments, wherein the RNA complex further comprises a strand that comprises a sequence having at least 80% identity to SEQ ID NO: 23.

Embodiment 46. The RNA complex of any of the above or below embodiments, wherein the RNA complex further comprises a strand that comprises a sequence of SEQ ID NO: 23.

Embodiment 47. The RNA complex of any of the above or below embodiments, wherein the RNA complex further comprises a strand that consists of a sequence of SEQ ID NO: 23.

Embodiment 48. The RNA complex of any of the above or below embodiments, wherein the strand consists of a sequence of SEQ ID NO: 4.

Embodiment 49. The RNA complex of any of the above or below embodiments, wherein the RNA complex further comprises a strand that comprises a sequence having at least 80% identity to SEQ ID NO: 23.

Embodiment 50. The RNA complex of any of the above or below embodiments, wherein the RNA complex further comprises a strand that comprises a sequence of SEQ ID NO: 23.

Embodiment 51. The RNA complex of any of the above or below embodiments, wherein the RNA complex further comprises a strand that consists of a sequence of SEQ ID NO: 23.

Embodiment 52. The RNA complex of any of the above or below embodiments, wherein the strand comprises a sequence having at least 80% identity to SEQ ID NO: 11.

Embodiment 53. The RNA complex of any of the above or below embodiments, wherein the strand includes at least one of: an overhang at the 5′ end of the sequence having at least 80% identity to SEQ ID NO: 11 or an overhang at the 3′ end of the sequence having at least 80% identity to SEQ ID NO: 11.

Embodiment 54. The RNA complex of any of the above or below embodiments, wherein the strand includes at least one of: adenosine monophosphate (rA) at the 5′ end of the sequence having at least 80% identity to SEQ ID NO: 11, uridine monophosphate-adenosine monophosphate (rU-rA) at the 5′ end of the sequence having at least 80% identity to SEQ ID NO: 11, double uridine monophosphate (rU-rU) at the 3′ end of the sequence having at least 80% identity to SEQ ID NO: 11, double deoxythymidine monophosphates (dT-dT) at the 3′ end of the sequence having at least 80% identity to SEQ ID NO: 11, 2′-O-methylated cytidine monophosphate-uridine monophosphate (oC-oU) at the 3′ end of the sequence having at least 80% identity to SEQ ID NO: 11, and double 2′-O-methyl-uridine monophosphates (oU-oU) at the 3′ end of the sequence having at least 80% identity to SEQ ID NO: 11.

Embodiment 55. The RNA complex of any of the above or below embodiments, wherein the RNA complex further comprises a strand that comprises a sequence having at least 80% identity to SEQ ID NO: 30.

Embodiment 56. The RNA complex of any of the above or below embodiments, wherein the RNA complex further comprises a strand that comprises a sequence of SEQ ID NO: 30.

Embodiment 57. The RNA complex of any of the above or below embodiments, wherein the RNA complex further comprises a strand that consists of a sequence of SEQ ID NO: 30.

Embodiment 58. The RNA complex of any of the above or below embodiments, wherein the strand comprises a sequence of SEQ ID NO: 11.

Embodiment 59. The RNA complex of any of the above or below embodiments, wherein the strand includes at least one of: an overhang at the 5′ end of the sequence of SEQ ID NO: 11 or an overhang at the 3′ end of the sequence of SEQ ID NO: 11.

Embodiment 60. The RNA complex of any of the above or below embodiments, wherein the strand includes at least one of: adenosine monophosphate (rA) at the 5′ end of the sequence of SEQ ID NO: 11, uridine monophosphate-adenosine monophosphate (rU-rA) at the 5′ end of the sequence of SEQ ID NO: 11, double uridine monophosphate (rU-rU) at the 3′ end of the sequence of SEQ ID NO: 11, double deoxythymidine monophosphates (dT-dT) at the 3′ end of the sequence of SEQ ID NO: 11, 2′-O-methylated cytidine monophosphate-uridine monophosphate (oC-oU) at the 3′ end of the sequence of SEQ ID NO: 11, and double 2′-O-methyl-uridine monophosphates (oU-oU) at the 3′ end of the sequence of SEQ ID NO: 11.

Embodiment 61. The RNA complex of any of the above or below embodiments, wherein the RNA complex further comprises a strand that comprises a sequence having at least 80% identity to SEQ ID NO: 30.

Embodiment 62. The RNA complex of any of the above or below embodiments, wherein the RNA complex further comprises a strand that comprises a sequence of SEQ ID NO: 30.

Embodiment 63. The RNA complex of any of the above or below embodiments, wherein the RNA complex further comprises a strand that consists of a sequence of SEQ ID NO: 30.

Embodiment 64. The RNA complex of any of the above or below embodiments, wherein the strand consists of a sequence of SEQ ID NO: 11.

Embodiment 65. The RNA complex of any of the above or below embodiments, wherein the RNA complex further comprises a strand that comprises a sequence having at least 80% identity to SEQ ID NO: 30.

Embodiment 66. The RNA complex of any of the above or below embodiments, wherein the RNA complex further comprises a strand that comprises a sequence of SEQ ID NO: 30.

Embodiment 67. The RNA complex of any of the above or below embodiments, wherein the RNA complex further comprises a strand that consists of a sequence of SEQ ID NO: 30.

Embodiment 68. The RNA complex of any of the above or below embodiments, wherein the strand comprises a sequence having at least 80% identity to SEQ ID NO: 16.

Embodiment 69. The RNA complex of any of the above or below embodiments, wherein the strand includes at least one of: an overhang at the 5′ end of the sequence having at least 80% identity to SEQ ID NO: 16 or an overhang at the 3′ end of the sequence having at least 80% identity to SEQ ID NO: 16.

Embodiment 70. The RNA complex of any of the above or below embodiments, wherein the strand includes at least one of: guanosine monophosphate (rG) at the 5′ end of the sequence having at least 80% identity to SEQ ID NO: 16, double guanosine monophosphate (rG-rG) at the 5′ end of the sequence having at least 80% identity to SEQ ID NO: 16, double uridine monophosphate (rU-rU) at the 3′ end of the sequence having at least 80% identity to SEQ ID NO: 16, double deoxythymidine monophosphates (dT-dT) at the 3′ end of the sequence having at least 80% identity to SEQ ID NO: 16, 2′-O-methylated guanosine monophosphate-cytidine monophosphate (oG-oC) at the 3′ end of the sequence having at least 80% identity to SEQ ID NO: 16, and double 2′-O-methyl-uridine monophosphates (oU-oU) at the 3′ end of the sequence having at least 80% identity to SEQ ID NO: 16.

Embodiment 71. The RNA complex of any of the above or below embodiments, wherein the RNA complex further comprises a strand that comprises a sequence having at least 80% identity to SEQ ID NO: 35.

Embodiment 72. The RNA complex of any of the above or below embodiments, wherein the RNA complex further comprises a strand that comprises a sequence of SEQ ID NO: 35.

Embodiment 73. The RNA complex of any of the above or below embodiments, wherein the RNA complex further comprises a strand that consists of a sequence of SEQ ID NO: 35.

Embodiment 74. The RNA complex of any of the above or below embodiments, wherein the strand comprises a sequence of SEQ ID NO: 16.

Embodiment 75. The RNA complex of any of the above or below embodiments, wherein the strand includes at least one of: an overhang at the 5′ end of the sequence of SEQ ID NO: 16 or an overhang at the 3′ end of the sequence of SEQ ID NO: 16.

Embodiment 76. The RNA complex of any of the above or below embodiments, wherein the strand includes at least one of: guanosine monophosphate (rG) at the 5′ end of the sequence of SEQ ID NO: 16, double guanosine monophosphate (rG-rG) at the 5′ end of the sequence of SEQ ID NO: 16, double uridine monophosphate (rU-rU) at the 3′ end of the sequence of SEQ ID NO: 16, double deoxythymidine monophosphates (dT-dT) at the 3′ end of the sequence of SEQ ID NO: 16, 2′-O-methylated guanosine monophosphate-cytidine monophosphate (oG-oC) at the 3′ end of the sequence of SEQ ID NO: 16, and double 2′-O-methyl-uridine monophosphates (oU-oU) at the 3′ end of the sequence of SEQ ID NO: 16.

Embodiment 77. The RNA complex of any of the above or below embodiments, wherein the RNA complex further comprises a strand that comprises a sequence having at least 80% identity to SEQ ID NO: 35.

Embodiment 78. The RNA complex of any of the above or below embodiments, wherein the RNA complex further comprises a strand that comprises a sequence of SEQ ID NO: 35.

Embodiment 79. The RNA complex of any of the above or below embodiments, wherein the RNA complex further comprises a strand that consists of a sequence of SEQ ID NO: 35.

Embodiment 80. The RNA complex of any of the above or below embodiments, wherein the strand consists of a sequence of SEQ ID NO: 16.

Embodiment 81. The RNA complex of any of the above or below embodiments, wherein the RNA complex further comprises a strand that comprises a sequence having at least 80% identity to SEQ ID NO: 35.

Embodiment 82. The RNA complex of any of the above or below embodiments, wherein the RNA complex further comprises a strand that comprises a sequence of SEQ ID NO: 35.

Embodiment 83. The RNA complex of any of the above or below embodiments, wherein the RNA complex further comprises a strand that consists of a sequence of SEQ ID NO: 35.

Embodiment 84. A ribonucleic acid (RNA) complex comprising a strand that comprises a sequence having at least 80% identity to one of SEQ ID NOs: 20-38.

Embodiment 85. The RNA complex of any of the above or below embodiments, wherein the strand comprises a sequence having at least 80% identity to SEQ ID NO: 23.

Embodiment 86. The RNA complex of any of the above or below embodiments, wherein the strand includes at least one of: an overhang at the 5′ end of the sequence having at least 80% identity to SEQ ID NO: 23 or an overhang at the 3′ end of the sequence having at least 80% identity to SEQ ID NO: 23.

Embodiment 87. The RNA complex of any of the above or below embodiments, wherein the strand comprises a sequence having at least 80% identity to SEQ ID NO: 30.

Embodiment 88. The RNA complex of any of the above or below embodiments, wherein the strand includes at least one of: an overhang at the 5′ end of the sequence having at least 80% identity to SEQ ID NO: 30 or an overhang at the 3′ end of the sequence having at least 80% identity to SEQ ID NO: 30.

Embodiment 89. The RNA complex of any of the above or below embodiments, wherein the strand comprises a sequence having at least 80% identity to SEQ ID NO: 35.

Embodiment 90. The RNA complex of any of the above or below embodiments, wherein the strand includes at least one of: an overhang at the 5′ end of the sequence having at least 80% identity to SEQ ID NO: 35 or an overhang at the 3′ end of the sequence having at least 80% identity to SEQ ID NO: 35.

Embodiment 91. A ribonucleic acid (RNA) complex comprising a strand having a sequence overlapping with a sequence of a transforming growth factor beta induced (TGFBI) protein messenger RNA (mRNA), the strand containing adenine at a position corresponding to the c. 371G>A single nucleotide polymorphism (SNP) in exon 4 of the TGFBI gene.

Embodiment 92. A ribonucleic acid (RNA) complex comprising a sense strand and an antisense strand, wherein at least one of the sense strand and the antisense strand comprises a dTdT overhang.

Embodiment 93. The RNA complex of any of the above or below embodiments, wherein the sense strand and the antisense strand each comprises a dTdT overhang.

Embodiment 94. The RNA complex of any of the above or below embodiments, wherein the sense strand comprises GG nucleotides as an overhang and the antisense strand comprises UC nucleotides as an overhang.

Embodiment 95. The RNA complex of any of the above or below embodiments, wherein (a) the sense strand comprises a series of repeating 2′-OMe; and (b) the antisense strand comprises 2′-OMe.

Embodiment 96. The RNA complex of any of the above or below embodiments, wherein (a) the sense strand comprises 15 bases and alternating patterns of 2′-OMe and 2′-F; and (b) the antisense strand comprises alternating patterns of 2′-OMe and 2′-F; wherein the RNA complex comprises additional phosphorothioate links on the 3′ and 5′ ends of both the sense strand and the antisense strand.

Embodiment 97. The RNA complex of any of the above or below embodiments, wherein (a) the sense strand comprises two units of 2′-OMe at the 5′ end and at least two 2′-OMe modifications at either U or G residues other than at position 9; and (b) the antisense strand comprises a single 2′-OMe at position 2 from the 5′ end, PS bonds in a dTdT overhand, and all pyrimidines replaced with 2′F-RNA units.

Embodiment 98. The RNA complex of any of the above or below embodiments, wherein the RNA complex includes a short interfering RNA duplex.

Embodiment 99. The RNA complex of any of the above or below embodiments, wherein the RNA complex includes a double stranded RNA complex configured for forming a short interfering RNA duplex.

Embodiment 100. The RNA complex of any of the above or below embodiments, wherein the RNA complex includes an RNA hairpin.

Embodiment 101. A method of preventing, ameliorating, or treating granular corneal dystrophy type 2 in a subject comprising administering to the subject any RNA complex described herein.

Embodiment 102. The method of any of the above or below embodiments, wherein the administering comprises injecting the RNA complex into the subject.

Embodiment 103. The method of any of the above or below embodiments, wherein the administering comprises applying a solution containing the RNA complex onto the subject.

Embodiment 104. The method of any of the above or below embodiments, wherein the administering comprises introducing the RNA complex into a cell containing and expressing a deoxyribonucleic acid (DNA) molecule having the c. 371G>A single nucleotide polymorphism (SNP) in exon 4 of a transforming growth factor beta induced (TGFBI) gene.

Embodiment 105. The method of any of the above or below embodiments, wherein the subject is a vertebrate.

Embodiment 106. The method of any of the above or below embodiments, wherein the subject is human.

Embodiment 107. The method of any of the above or below embodiments, wherein the method further comprises: prior to administering to the subject the RNA complex: obtaining sequence information of the subject; and determining that the subject has an allele having the c. 371G>A SNP in exon 4 of the TGFBI gene and an allele that does not have the c. 371G>A SNP in exon 4 of the TGFBI gene.

Embodiment 108. The method of any of the above or below embodiments, wherein the sequence information of the subject consists of sequence information of exon 4 of the TGFBI gene.

Embodiment 109. The method of any of the above or below embodiments, wherein the sequence information of the subject includes sequence information of a subset, less than all, of exon 4 of the TGFBI gene.

Embodiment 110. The method of any of the above or below embodiments, wherein the sequence information of the subject includes only the sequence information of the c. 371G>A SNP in exon 4 of the TGFBI gene.

Embodiment 111. The method of any of the above or below embodiments, wherein the sequence information of the subject includes whole-genome sequence information of the subject.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein can be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially effect the basic and novel characteristic(s). Embodiments of the disclosure so claimed are inherently or expressly described and enabled herein.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims. All references, patents, and patent applications referred to in this application are herein incorporated by reference in their entirety.

1. A ribonucleic acid (RNA) complex comprising a strand that comprises a sequence having at least 80% identity to one of SEQ ID NOs: 1-19.

2. The RNA complex of claim 1, wherein the strand comprises a sequence having at least 80% identity to SEQ ID NO: 4, 11 or 16.

3. The RNA complex of claim 2, wherein the strand includes at least one of: an overhang at the 5′ end of the sequence having at least 80% identity to SEQ ID NO: 4, 11, or 16 or an overhang at the 3′ end of the sequence having at least 80% identity to SEQ ID NO: 4, 11, or 16.

4. The RNA complex of claim 3, wherein the strand includes at least one of: cytidine monophosphate (rC) at the 5′ end of the sequence having at least 80% identity to SEQ ID NO: 4, 11, or 16, uridine monophosphate-cytidine monophosphate (rU-rC) at the 5′ end of the sequence having at least 80% identity to SEQ ID NO: 4, 11, or 16, double uridine monophosphate (rU-rU) at the 3′ end of the sequence having at least 80% identity to SEQ ID NO: 4, 11, or 16, double deoxythymidine monophosphates (dT-dT) at the 3′ end of the sequence having at least 80% identity to SEQ ID NO: 4, 11, or 16, double 2′-O-methylated guanosine monophosphates (oG-oG) at the 3′ end of the sequence having at least 80% identity to SEQ ID NO: 4, 11, or 16, and double 2′-O-methyl-uridine monophosphates (oU-oU) at the 3′ end of the sequence having at least 80% identity to SEQ ID NO: 4, 11, or 16.

5. The RNA complex of claim 2, further comprising a strand that comprises a sequence having at least 80% identity to SEQ ID NO: 23, 30 or 35.

6. The RNA complex of claim 2, further comprising a strand that comprises a sequence of SEQ ID NO: 23, 30 or 35.

7. The RNA complex of claim 1, wherein the strand comprises a sequence of SEQ ID NO: 4, 11, or 16.

8. The RNA complex of claim 1, wherein the RNA complex comprises a first strand and a second strand, each comprising at least one TGFBI R124H mutation site compared to a wild-type 124C TGFBI gene.

9. The RNA complex of claim 8, wherein the first strand comprises a sequence that is identical to the sequence of SEQ ID NO: 4, 9 or 11, except for one base that is mismatched to a juxtaposed base in the second strand.

10. The RNA complex of claim 9, wherein the mismatched base is between three and seven bases away from the TGFBI R124H mutation site.

11. The RNA complex of claim 8, wherein the first strand and the second strand are each between 16 and 23 bases in length.

12. The RNA complex of claim 8, wherein the RNA complex has deoxythymidine overhangs.

13. A ribonucleic acid (RNA) complex comprising a strand that comprises a sequence having at least 80% identity to one of SEQ ID NOs: 20-38.

14. The RNA complex of claim 13, wherein the strand comprises a sequence having at least 80% identity to SEQ ID NO: 23, 30 or 35.

15. The RNA complex of claim 13, wherein the strand includes at least one of: an overhang at the 5′ end of the sequence having at least 80% identity to SEQ ID NO: 23, 30 or 35 or an overhang at the 3′ end of the sequence having at least 80% identity to SEQ ID NO: 23, 30 or 35.

16. A ribonucleic acid (RNA) complex comprising a strand having a sequence overlapping with a sequence of a transforming growth factor beta induced (TGFBI) protein messenger RNA (mRNA), the strand containing adenine at a position corresponding to the c. 371G>A single nucleotide polymorphism (SNP) in exon 4 of the TGFBI gene.

17. A ribonucleic acid (RNA) complex comprising a sense strand and an antisense strand, wherein at least one of the sense strand and the antisense strand comprises a dTdT overhang.

18. The RNA complex of claim 17, wherein the sense strand and the antisense strand each comprises a dTdT overhang.

19. The RNA complex of claim 17, wherein the sense strand comprises GG nucleotides as an overhang and the antisense strand comprises UC nucleotides as an overhang.

20. The RNA complex of claim 17, wherein
(a) the sense strand comprises a series of repeating 2′-OMe; and (b) the antisense strand comprises 2′-OMe;
(a) the sense strand comprises 15 bases and alternating patterns of 2′-OMe and 2′-F; and
(b) the antisense strand comprises alternating patterns of 2′-OMe and 2′-F, wherein the RNA complex comprises additional phosphorothioate links on the 3′ and 5′ ends of both the sense strand and the antisense strand; or
(a) the sense strand comprises two units of 2′-OMe at the 5′ end and at least two 2′-OMe modifications at either U or G residues other than at position 9; and (b) the antisense strand comprises a single 2′-OMe at position 2 from the 5′ end, PS bonds in a dTdT overhand, and all pyrimidines replaced with 2′F-RNA units.

21. The RNA complex of claim 1, wherein the RNA complex includes a short interfering RNA duplex; a double stranded RNA complex configured for forming a short interfering RNA duplex; and/or an RNA hairpin.

22. A method of preventing, ameliorating, or treating granular corneal dystrophy type 2 in a subject, the method comprising:
administering to the subject the RNA complex of claim 1.

23. The method of claim 22, wherein the administering comprises introducing the RNA complex into a cell containing and expressing a deoxyribonucleic acid (DNA) molecule having the c. 371G>A single nucleotide polymorphism (SNP) in exon 4 of a transforming growth factor beta induced (TGFBI) gene.

24. The method of claim 22, further comprising:
prior to administering to the subject the RNA complex:
obtaining sequence information of the subject; and
determining that the subject has an allele having the c. 371G>A SNP in exon 4 of the TGFBI gene and an allele that does not have the c. 371G>A SNP in exon 4 of the TGFBI gene.


SEQUENCE LISTING (110 sequences)

SEQ ID NO: 1 - RNA, 19 nt, Artificial Sequence
  synthetic polymer
cucagcuguacacggacca

SEQ ID NO: 2 - RNA, 19 nt, Artificial Sequence
  synthetic polymer
ucagcuguacacggaccac

SEQ ID NO: 3 - RNA, 19 nt, Artificial Sequence
  synthetic polymer
cagcuguacacggaccaca

SEQ ID NO: 4 - RNA, 19 nt, Artificial Sequence
  synthetic polymer
agcuguacacggaccacac

SEQ ID NO: 5 - RNA, 19 nt, Artificial Sequence
  synthetic polymer
gcuguacacggaccacacg

SEQ ID NO: 6 - RNA, 19 nt, Artificial Sequence
  synthetic polymer
cuguacacggaccacacgg

SEQ ID NO: 7 - RNA, 19 nt, Artificial Sequence
  synthetic polymer
uguacacggaccacacgga

SEQ ID NO: 8 - RNA, 19 nt, Artificial Sequence
  synthetic polymer
guacacggaccacacggag

SEQ ID NO: 9 - RNA, 19 nt, Artificial Sequence
  synthetic polymer
uacacggaccacacggaga

SEQ ID NO: 10 - RNA, 19 nt, Artificial Sequence
  synthetic polymer
acacggaccacacggagaa

SEQ ID NO: 11 - RNA, 19 nt, Artificial Sequence
  synthetic polymer
cacggaccacacggagaag

SEQ ID NO: 12 - RNA, 19 nt, Artificial Sequence
  synthetic polymer
acggaccacacggagaagc

SEQ ID NO: 13 - RNA, 19 nt, Artificial Sequence
  synthetic polymer
cggaccacacggagaagcu

SEQ ID NO: 14 - RNA, 19 nt, Artificial Sequence
  synthetic polymer
ggaccacacggagaagcug

SEQ ID NO: 15 - RNA, 19 nt, Artificial Sequence
  synthetic polymer
gaccacacggagaagcuga

SEQ ID NO: 16 - RNA, 19 nt, Artificial Sequence
  synthetic polymer
accacacggagaagcugag

SEQ ID NO: 17 - RNA, 19 nt, Artificial Sequence
  synthetic polymer
ccacacggagaagcugagg

SEQ ID NO: 18 - RNA, 19 nt, Artificial Sequence
  synthetic polymer
cacacggagaagcugaggc

SEQ ID NO: 19 - RNA, 19 nt, Artificial Sequence
  synthetic polymer
acacggagaagcugaggcc

SEQ ID NO: 20 - RNA, 19 nt, Artificial Sequence
  synthetic polymer
ugguccguguacagcugag

SEQ ID NO: 21 - RNA, 19 nt, Artificial Sequence
  synthetic polymer
gugguccguguacagcuga

SEQ ID NO: 22 - RNA, 19 nt, Artificial Sequence
  synthetic polymer
ugugguccguguacagcug

SEQ ID NO: 23 - RNA, 19 nt, Artificial Sequence
  synthetic polymer
gugugguccguguacagcu

SEQ ID NO: 24 - RNA, 19 nt, Artificial Sequence
  synthetic polymer
cgugugguccguguacagc

SEQ ID NO: 25 - RNA, 19 nt, Artificial Sequence
  synthetic polymer
ccgugugguccguguacag

SEQ ID NO: 26 - RNA, 19 nt, Artificial Sequence
  synthetic polymer
uccgugugguccguguaca

SEQ ID NO: 27 - RNA, 19 nt, Artificial Sequence
  synthetic polymer
cuccgugugguccguguac

SEQ ID NO: 28 - RNA, 19 nt, Artificial Sequence
  synthetic polymer
ucuccgugugguccgugua

SEQ ID NO: 29 - RNA, 19 nt, Artificial Sequence
  synthetic polymer
uucuccgugugguccgugu

SEQ ID NO: 30 - RNA, 19 nt, Artificial Sequence
  synthetic polymer
cuucuccgugugguccgug

SEQ ID NO: 31 - RNA, 19 nt, Artificial Sequence
  synthetic polymer
gcuucuccgugugguccgu

SEQ ID NO: 32 - RNA, 19 nt, Artificial Sequence
  synthetic polymer
agcuucuccgugugguccg

SEQ ID NO: 33 - RNA, 19 nt, Artificial Sequence
  synthetic polymer
cagcuucuccguguggucc

SEQ ID NO: 34 - RNA, 19 nt, Artificial Sequence
  synthetic polymer
ucagcuucuccgugugguc

SEQ ID NO: 35 - RNA, 19 nt, Artificial Sequence
  synthetic polymer
cucagcuucuccguguggu

SEQ ID NO: 36 - RNA, 19 nt, Artificial Sequence
  synthetic polymer
ccucagcuucuccgugugg

SEQ ID NO: 37 - RNA, 19 nt, Artificial Sequence
  synthetic polymer
gccucagcuucuccgugug

SEQ ID NO: 38 - RNA, 19 nt, Artificial Sequence
  synthetic polymer
ggccucagcuucuccgugu

SEQ ID NO: 39 - RNA, 19 nt, Artificial Sequence
  synthetic polymer
uagcgacuaaacacaucaa

SEQ ID NO: 40 - RNA, 19 nt, Artificial Sequence
  synthetic polymer
gugcguugcuaguaccaac

SEQ ID NO: 41 - RNA, 19 nt, Artificial Sequence
  synthetic polymer
uugauguguuuagucgcua

SEQ ID NO: 42 - RNA, 19 nt, Artificial Sequence
  synthetic polymer
guugguacuagcaacgcac

SEQ ID NO: 43 - DNA, 37 bp, Artificial Sequence
  synthetic polymer
ctcagctgtacacggaccgcacggagaagctgaggcc

SEQ ID NO: 44 - DNA, 37 bp, Artificial Sequence
  synthetic polymer
ctcagctgtacacggaccacacggagaagctgaggcc

SEQ ID NO: 45 - RNA, 20 nt, Artificial Sequence
  synthetic polymer
acacggaccacagggagatt

SEQ ID NO: 46 - RNA, 21 nt, Artificial Sequence
  synthetic polymer
cacggaccacagggagaagtt

SEQ ID NO: 47 - RNA, 20 nt, Artificial Sequence
  synthetic polymer
acacggaccacacgcagatt

SEQ ID NO: 48 - RNA, 21 nt, Artificial Sequence
  synthetic polymer
cacggaccacacgcagaagtt

SEQ ID NO: 49 - RNA, 20 nt, Artificial Sequence
  synthetic polymer
acacggaccacacggacatt

SEQ ID NO: 50 - RNA, 21 nt, Artificial Sequence
  synthetic polymer
cacggaccacacggacaagtt

SEQ ID NO: 51 - DNA, 37 bp, Artificial Sequence; identical to SEQ ID NO: 43
  synthetic polymer

SEQ ID NO: 52 - DNA, 37 bp, Artificial Sequence; identical to SEQ ID NO: 44
  synthetic polymer

SEQ ID NO: 53 - RNA, 18 nt, Artificial Sequence
  synthetic polymer
ccagcgacacggaccatt

SEQ ID NO: 54 - RNA, 18 nt, Artificial Sequence
  synthetic polymer
cagcgacacggaccactt

SEQ ID NO: 55 - RNA, 19 nt, Artificial Sequence
  synthetic polymer
cagcgacacggaccacatt

SEQ ID NO: 56 - RNA, 19 nt, Artificial Sequence
  synthetic polymer
agcgacacggaccacactt

SEQ ID NO: 57 - RNA, 19 nt, Artificial Sequence
  synthetic polymer
gcgacacggaccacacgtt

SEQ ID NO: 58 - RNA, 19 nt, Artificial Sequence
  synthetic polymer
cgacacggaccacacggtt

SEQ ID NO: 59 - RNA, 19 nt, Artificial Sequence
  synthetic polymer
gacacggaccacacggatt

SEQ ID NO: 60 - RNA, 20 nt, Artificial Sequence
  synthetic polymer
gacacggaccacacggagtt

SEQ ID NO: 61 - RNA, 20 nt, Artificial Sequence
  synthetic polymer
acacggaccacacggagatt

SEQ ID NO: 62 - RNA, 21 nt, Artificial Sequence
  synthetic polymer
acacggaccacacggagaatt

SEQ ID NO: 63 - RNA, 21 nt, Artificial Sequence
  synthetic polymer
cacggaccacacggagaagtt

SEQ ID NO: 64 - RNA, 21 nt, Artificial Sequence
  synthetic polymer
acggaccacacggagaagctt

SEQ ID NO: 65 - RNA, 20 nt, Artificial Sequence
  synthetic polymer
cggaccacacggagaagctt

SEQ ID NO: 66 - RNA, 20 nt, Artificial Sequence
  synthetic polymer
ggaccacacggagaagcgtt

SEQ ID NO: 67 - RNA, 20 nt, Artificial Sequence
  synthetic polymer
gaccacacggagaagcgatt

SEQ ID NO: 68 - RNA, 20 nt, Artificial Sequence
  synthetic polymer
accacacggagaagcgagtt

SEQ ID NO: 69 - RNA, 20 nt, Artificial Sequence
  synthetic polymer
ccacacggagaagcgaggtt

SEQ ID NO: 70 - RNA, 20 nt, Artificial Sequence
  synthetic polymer
cacacggagaagcgaggctt

SEQ ID NO: 71 - RNA, 20 nt, Artificial Sequence
  synthetic polymer
acacggagaagcgaggcctt

SEQ ID NO: 72 - RNA, 21 nt, Artificial Sequence
  synthetic polymer
cgacaagccggcgcagadtdt

SEQ ID NO: 73 - RNA, 16 nt, Artificial Sequence
  synthetic polymer
acgcgccaggcgcgtt

SEQ ID NO: 74 - RNA, 19 nt, Artificial Sequence
  synthetic polymer
cgacaagccggcgcagagg

SEQ ID NO: 75 - RNA, 15 nt, Artificial Sequence
  synthetic polymer
acgcgccaggcgcgc

SEQ ID NO: 76 - RNA, 19 nt, Artificial Sequence
  synthetic polymer
cgacaagccggcgcagatt

SEQ ID NO: 77 - RNA, 16 nt, Artificial Sequence
  synthetic polymer
acgcgccaggcgcgtt

SEQ ID NO: 78 - RNA, 13 nt, Artificial Sequence
  synthetic polymer
aagccggcgcaga

SEQ ID NO: 79 - RNA, 14 nt, Artificial Sequence
  synthetic polymer
acgcgccaggcgcg

SEQ ID NO: 80 - RNA, 19 nt, Artificial Sequence
  synthetic polymer
cgacaagccggcgcagatt

SEQ ID NO: 81 - RNA, 16 nt, Artificial Sequence
  synthetic polymer
acgcgccaggcgcgtt

SEQ ID NO: 82 - DNA, 37 bp, Artificial Sequence; identical to SEQ ID NO: 43
  synthetic polymer

SEQ ID NO: 83 - DNA, 37 bp, Artificial Sequence; identical to SEQ ID NO: 44
  synthetic polymer

SEQ ID NO: 84 - RNA, 19 nt, Artificial Sequence
  synthetic polymer
agcgacacggaccacactt

SEQ ID NO: 85 - RNA, 19 nt, Artificial Sequence
  synthetic polymer
agcgacacggaccacagtt

SEQ ID NO: 86 - RNA, 20 nt, Artificial Sequence
  synthetic polymer
acacggaccacacggagatt

SEQ ID NO: 87 - RNA, 20 nt, Artificial Sequence
  synthetic polymer
acacggaccacagggagatt

SEQ ID NO: 88 - RNA, 20 nt, Artificial Sequence
  synthetic polymer
acacggaccacacgcagatt

SEQ ID NO: 89 - RNA, 20 nt, Artificial Sequence
  synthetic polymer
acacggaccacacggacatt

SEQ ID NO: 90 - RNA, 21 nt, Artificial Sequence
  synthetic polymer
cacggaccacacggagaagtt

SEQ ID NO: 91 - RNA, 21 nt, Artificial Sequence
  synthetic polymer
cacggaccacagggagaagtt

SEQ ID NO: 92 - RNA, 21 nt, Artificial Sequence
  synthetic polymer
cacggaccacacgcagaagtt

SEQ ID NO: 93 - RNA, 21 nt, Artificial Sequence
  synthetic polymer
cacggaccacacggacaagtt

SEQ ID NO: 94 - DNA, 37 bp, Artificial Sequence; identical to SEQ ID NO: 43
  synthetic polymer

SEQ ID NO: 95 - DNA, 37 bp, Artificial Sequence; identical to SEQ ID NO: 44
  synthetic polymer

SEQ ID NO: 96 - RNA, 19 nt, Artificial Sequence
  synthetic polymer
agcgacacggaccacactt

SEQ ID NO: 97 - RNA, 20 nt, Artificial Sequence
  synthetic polymer
cagcgacacggaccacactt

SEQ ID NO: 98 - RNA, 21 nt, Artificial Sequence
  synthetic polymer
tcagcgacacggaccacactt

SEQ ID NO: 99 - RNA, 22 nt, Artificial Sequence
  synthetic polymer
ctcagcgacacggaccacactt

SEQ ID NO: 100 - RNA, 20 nt, Artificial Sequence
  synthetic polymer
agcgacacggaccacacgtt

SEQ ID NO: 101 - RNA, 21 nt, Artificial Sequence
  synthetic polymer
agcgacacggaccacacggtt

SEQ ID NO: 102 - RNA, 22 nt, Artificial Sequence
  synthetic polymer
agcgacacggaccacacggatt

SEQ ID NO: 103 - RNA, 18 nt, Artificial Sequence
  synthetic polymer
gcgacacggaccacactt

SEQ ID NO: 104 - RNA, 17 nt, Artificial Sequence
  synthetic polymer
cgacacggaccacactt

SEQ ID NO: 105 - RNA, 16 nt, Artificial Sequence
  synthetic polymer
gacacggaccacactt

SEQ ID NO: 106 - RNA, 18 nt, Artificial Sequence
  synthetic polymer
agcgacacggaccacatt

SEQ ID NO: 107 - RNA, 17 nt, Artificial Sequence
  synthetic polymer
agcgacacggaccactt

SEQ ID NO: 108 - RNA, 16 nt, Artificial Sequence
  synthetic polymer
agcgacacggaccatt

SEQ ID NO: 109 - RNA, 21 nt, Artificial Sequence
  synthetic polymer
cagcgacacggaccacacgtt

SEQ ID NO: 110 - RNA, 23 nt, Artificial Sequence
  synthetic polymer
tcagcgacacggaccacacggtt